(19) United States
(12) Patent Application Publication
(10) Pub. No.: US 2026/0110100 A1
(43) Pub. Date: Apr. 23, 2026

(54) SYSTEMS AND METHODS FOR INTEGRATED MODULAR PRODUCTION OF HYDROGEN FRESHWATER, AND BIOELECTRICITY

(71) Applicants: Mohammadali KHALVATI, Newmarket (CA); Ibrahim DINCER, Whitby (CA); Aysegul Yagmur GOREN KARA, Oshawa (CA); Ahmet KILICASLAN, Oshawa (CA)

(72) Inventors: Mohammadali KHALVATI, Newmarket (CA); Ibrahim DINCER, Whitby (CA); Aysegul Yagmur GOREN KARA, Oshawa (CA); Ahmet KILICASLAN, Oshawa (CA)

(21) Appl. No.: 19/358,638

(22) Filed: Oct. 15, 2025

Related U.S. Application Data
(60) Provisional application No. 63/708,465, filed on Oct. 17, 2024.

(51) Int. Cl. C25B 5/00; B01D 61/46; B09B 3/65; C02F 1/469; C12M 1/00; C12M 3/00; C25B 1/04; C25B 9/17; H01M 8/04537; H01M 8/16

(52) U.S. Cl. CPC C25B 5/00; B01D 61/461; B09B 3/65; C02F 1/4693; C12M 23/44; C12M 43/00; C25B 1/04; C25B 9/17; H01M 8/04552; H01M 8/16; B01D 2313/02; B01D 2313/041; C02F 2201/004

(57) ABSTRACT

Provided are systems and methods for multi-process generators employing fermentation, desalination, and electrolysis technologies. The generator system includes a fermentation compartment configured to receive a mixture of biomass waste and an anaerobic microorganism solution comprising bacteria for bioenergy production; an electrolysis compartment configured to receive an electrolyte solution comprising a saline mixture, the electrolysis compartment including first and second spaced apart electrodes at least partially submerged in the electrolyte solution; and a desalination compartment positioned between the fermentation compartment and the electrolysis compartment, the desalination compartment configured to receive a saline solution and comprising an anion exchange membrane separating the desalination compartment from the electrolysis compartment and a cation exchange membrane separating the desalination compartment from the fermentation compartment, wherein the desalination compartment is configured to perform ion exchange processes to produce freshwater.

### CROSS-REFERENCE TO RELATED APPLICATION(S)

This application claims benefit of U.S. Provisional Patent Application No. 63/708,465 filed on Oct. 17, 2024 entitled “SYSTEMS AND METHODS FOR INTEGRATED MODULAR PRODUCTION OF HYDROGEN, FRESHWATER, AND BIOELECTRICITY”. The content of U.S. 63/708,465 is incorporated herein by reference in its entirety.

### FIELD

The embodiments described herein generally relate to systems and methods for integrated modular production of hydrogen, freshwater, and bioelectricity and, in particular, to multi-process generators employing fermentation, desalination, and electrolysis technologies.

### BACKGROUND

The following is not an admission that anything discussed below is part of the prior art or part of the common general knowledge of a person skilled in the art.

The global demand for sustainable energy sources and clean water has driven research and development efforts in various technological fields, particularly those related to hydrogen production, desalination, and renewable energy generation. Hydrogen, often termed the “fuel of the future,” has gained attention as a clean and efficient energy carrier that can be used in fuel cells, transportation, and various industrial applications.

Freshwater, an essential resource for human survival and industrial processes, is increasingly scarce due to population growth, climate change, and pollution. Additionally, bioelectricity, derived from biological processes, offers a renewable and sustainable source of energy, which is crucial in reducing reliance on fossil fuels and minimizing environmental impact.

Traditional methods of hydrogen production, such as steam methane reforming, and water electrolysis, have been widely implemented. However, these processes are often energy-intensive, costly, and generate significant greenhouse gas emissions. The production of freshwater, primarily through conventional desalination methods like reverse osmosis and thermal distillation, also presents challenges, including high energy consumption, environmental concerns related to brine disposal, and the extensive infrastructure required. Bioelectricity generation, commonly through microbial fuel cells, faces challenges related to efficiency, scalability, and the need for continuous and reliable bio-catalysts.

The existing technologies for hydrogen production, freshwater generation, and bioelectricity production have generally been developed and operated as separate systems. This separation often results in inefficiencies, as each system requires its own energy input, infrastructure, and maintenance. Moreover, the focus has largely been on improving individual processes rather than integrating them into a unified system that can simultaneously address the demand for hydrogen, freshwater, and renewable energy. Such a lack of integration limits the potential for optimizing resource utilization and reducing the overall environmental footprint of these processes.

Another challenge with current technologies is the restricted ability to easily scale or adapt the systems to varying production needs. For example, hydrogen production systems designed for large-scale industrial applications may not be easily modified for smaller, community-based operations or for integration with renewable energy sources. Similarly, traditional desalination plants are often too large and expensive for small-scale or remote applications, where water scarcity is most acute. Bioelectricity generation systems, while promising, often lack the modularity and flexibility required to efficiently scale or integrate with other systems, limiting their broader adoption.

Additionally, the management of by-products and waste streams poses a challenge in existing hydrogen, desalination, and bioelectricity systems. For instance, the brine generated from desalination can have severe environmental impacts if not properly managed, and the carbon by-products from certain hydrogen production processes need to be carefully handled to avoid environmental harm. The bioelectricity systems also produce waste products that must be managed effectively to prevent system contamination and ensure long-term operational efficiency.

Accordingly, there is a need for an integrated solution that can address these challenges by combining hydrogen production, freshwater generation, and bioelectricity production. Such a system would optimize the use of resources, reduce energy consumption, minimize waste, and provide a scalable, adaptable solution that can be tailored to specific needs and applications. This approach would not only improve efficiency and sustainability but also make these technologies more accessible and viable for a wider range of applications, from large-scale industrial operations to small, remote communities.

### SUMMARY

This summary is intended to introduce the reader to the more detailed description that follows and not to limit or define any claimed or as yet unclaimed invention. One or more inventions may reside in any combination or sub-combination of the elements or process steps disclosed in any part of this document including its claims and figures.

In a first aspect, in at least one embodiment, there is provided a modular three-chambered cylindrical generator system. The system comprises a fermentation compartment configured to receive a mixture of biomass waste and an anaerobic microorganism solution comprising bacteria for bioenergy production, wherein the fermentation compartment includes first and second spaced apart electrodes at least partially immersed in the mixture; an electrolysis compartment configured to receive an electrolyte solution comprising a saline mixture, the electrolysis compartment including first and second spaced apart electrodes at least partially submerged in the electrolyte solution, the first and second spaced apart electrodes connected to an external electrical circuit through an applied external resistance; and a desalination compartment positioned between the fermentation compartment and the electrolysis compartment, the desalination compartment configured to receive a saline solution and comprising an anion exchange membrane separating the desalination compartment from the electrolysis compartment and a cation exchange membrane separating the desalination compartment from the fermentation compartment, wherein the desalination compartment is configured to perform ion exchange processes to produce freshwater.

In one or more embodiments, the first and second electrodes in the electrolysis compartment are spaced apart by a distance of approximately 5 to 10 centimeters.

In one or more embodiments, the electrodes in the fermentation compartment are connected to an external multimeter configured to measure the bioelectricity potential.

In one or more embodiments, the system further comprises mixers selected from the group consisting of paddle-type mixers, spiral mixers, and ribbon mixers, wherein the mixers are placed horizontally within the fermentation compartment and the electrolysis compartment.

In one or more embodiments, the fermentation compartment, the desalination compartment, and the electrolysis compartment are clamped together using gaskets and O-rings secured by stainless steel bolts to provide a leak-proof operation.

In another aspect, in at least one embodiment, there is provided a modular cascaded generator system. The system comprises an electrolysis compartment configured to receive an electrolyte solution comprising a saline mixture, the electrolysis compartment including first and second electrodes spaced apart and positioned parallel to each other on movable rails, the first and second electrodes connected to an external electrical circuit through an applied external resistance; a fermentation compartment configured to receive a mixture of biomass waste and an anaerobic microorganism solution comprising bacteria for bioenergy production, wherein the fermentation compartment includes first and second electrodes spaced apart and positioned parallel to each other on movable rails, the first and second electrodes connected to an external multimeter for measuring bioelectricity potential; and a desalination compartment positioned between the electrolysis compartment and the fermentation compartment, the desalination compartment configured to receive a saline solution and comprising an anion exchange membrane separating the desalination compartment from the electrolysis compartment and a cation exchange membrane separating the desalination compartment from the fermentation compartment, wherein the desalination compartment is configured to perform ion exchange processes to produce freshwater; wherein the compartments are clamped together using gaskets and O-rings secured by stainless steel bolts to provide a leak-proof operation.

In one or more embodiments, the first and second electrodes in the electrolysis compartment are spaced apart by a distance of approximately 5 to 10 centimeters.

In one or more embodiments, the electrolysis compartment further comprises multiple electrodes placed within the electrolysis compartment.

In one or more embodiments, the fermentation compartment is located above the desalination compartment and the hydrogen gas produced in the fermentation compartment is collected through a hole positioned at the top of the fermentation compartment.

In one or more embodiments, the desalination compartment includes an input located at the top of the desalination compartment for solution feeding and an output located at the bottom of the desalination compartment for solution collection.

In another aspect, in at least one embodiment, there is provided a modular intertwined generator system for the concurrent production of natural hydrogen, freshwater, and bioelectricity. The system comprises an electrolysis chamber located in an interior of the generator system, the electrolysis chamber including cylindrical electrodes spaced apart and separated from a desalination chamber by a cation exchange membrane, the cylindrical electrodes connected to an external electrical circuit via an applied external resistance, wherein the electrolysis chamber is configured to receive an electrolyte solution and hydrogen gas produced during the electrolysis process; a desalination chamber positioned in the middle of the generator system, the desalination chamber separated from the electrolysis chamber by the cation exchange membrane and from a fermentation chamber by an anion exchange membrane, the desalination chamber configured to receive a saline solution; and a fermentation chamber located at the outermost part of the generator system, the fermentation chamber including first and second spaced apart electrodes placed in a biomass solution for bioenergy collection, the electrodes connected to an external multimeter for measuring the bioelectricity potential.

In one or more embodiments, the cylindrical electrodes in the electrolysis chamber are spaced apart by a distance of approximately 5 to 10 centimeters.

In one or more embodiments, the generator system is constructed in a geometrical shape selected from a group of cylindrical, cubic, triangular, rectangular, square, pentagonal, hexagonal, heptagonal, octagonal, and trapezoidal.

In another aspect, in at least one embodiment, there is provided a method for generating electricity, desalinating water, and fermenting biomass within a modular three-chambered generator system. The method comprises initiating an electrolysis process within an electrolysis compartment to generate hydrogen gas and bioelectricity, wherein the electrolysis compartment comprises a saline mixture with sodium and chlorine ions; processing biomass in a fermentation chamber to produce hydrogen gas and bioelectricity, wherein the fermentation chamber comprises a mixture of biomass waste and an anaerobic microorganism solution; desalinating water in a desalination chamber positioned between the electrolysis chamber and fermentation chamber, wherein the desalinating chamber comprises a saline solution; and synchronizing, by an electronic device, the electrolysis, fermentation, and desalination processes, by monitoring and adjusting the flow rates, electrical inputs, and microbial activity across compartments.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment, and in which:

FIG. 1 is a system diagram of a modular three-chambered cylindrical natural hydrogen, freshwater, and bioelectricity generator system 100 in accordance with an embodiment.

FIG. 2 is a system diagram of a modular three-chambered cubic cylindrical natural hydrogen, freshwater, and bioelectricity generator system 200 in accordance with an embodiment.

FIG. 3 is a system diagram of a modular three-chambered triangular natural hydrogen, freshwater, and bioelectricity generator system 300 in accordance with an embodiment.

FIG. 4 is a system diagram of a modular three-chambered rectangular natural hydrogen, freshwater, and bioelectricity generator system 400 in accordance with an embodiment.

FIG. 5 is a system diagram of a modular three-chambered pentagonal natural hydrogen, freshwater, and bioelectricity generator system 500 in accordance with an embodiment.

FIG. 6 is a system diagram of an integrated modular cascaded generator system 600 in accordance with an embodiment.

FIG. 7 is a system diagram of an integrated modular cascaded generator system 700 in accordance with an embodiment.

FIG. 8 is a system diagram of an integrated modular cascaded square generator system 800 in accordance with an embodiment.

FIG. 9 is a system diagram of an integrated modular cascaded pentagonal generator system 900 in accordance with an embodiment.

FIG. 10 is a system diagram of an integrated modular cascaded hexagonal generator system 1000 in accordance with an embodiment.

FIG. 11 is a system diagram of an integrated modular cascaded heptagonal generator system 1100 in accordance with an embodiment.

FIG. 12 is a system diagram of an integrated modular cascaded octagonal generator system 1200 in accordance with an embodiment.

FIG. 13 is a system diagram of an integrated modular cascaded rectangular generator system 1300 in accordance with an embodiment.

FIG. 14 is a system diagram of a reactor system 1400 in accordance with an embodiment.

FIG. 15 is a system diagram of a reactor system 1500 in accordance with an embodiment.

FIG. 16 is a system diagram of an integrated modular intertwined generator system 1600 in accordance with an embodiment.

FIG. 17 is a cross-section view of an integrated modular intertwined generator system 1700 in accordance with an embodiment.

FIG. 18 is a system diagram of a membrane holder 1800 in accordance with an embodiment.

FIG. 19 is a system diagram of a multi-chamber generator system 1900 in accordance with an embodiment.

FIG. 20A and FIG. 20B are system diagrams of a modular single cell 2000 and a conductive rod in accordance with an embodiment.

FIG. 21A and FIG. 21B are system diagrams of a modular cascaded electrode cell 2100 and plate-type electrodes in accordance with an embodiment.

FIG. 22A and FIG. 22B are system diagrams of a modular cascaded bioenergy collector 2200 in accordance with an embodiment.

FIG. 23 is a system diagram of a modular water and sludge feeding unit 2300 in accordance with an embodiment.

FIG. 24A and FIG. 24B are system diagrams of a modular hydrogen gas collector 2400 in accordance with an embodiment.

FIG. 25A and FIG. 25B are system diagrams of a modular sludge collection and mixing device 2500 in accordance with an embodiment.

FIG. 26 is a flowchart 2600 of an example method for producing natural hydrogen gas in a fermentation chamber according to an embodiment.

FIG. 27 is a flowchart 2700 of an example method for assembling a modular three-chambered generator system according to an embodiment.

FIG. 28 is a flowchart 2800 of an example method for generating electricity, desalinating water, and fermenting biomass within a modular three-chambered generator system according to an embodiment.

The skilled person in the art will understand that the drawings, described below, are for illustration purposes only. The drawings are not intended to limit the scope of the applicants' teachings in any way. Also, it will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DESCRIPTION OF VARIOUS EMBODIMENTS

It will be appreciated that numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way, but rather as merely describing the implementation of the various embodiments described herein.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

The terms “including,” “comprising” and variations thereof mean “including but not limited to,” unless expressly specified otherwise. A listing of items does not imply that any or all of the items are mutually exclusive, unless expressly specified otherwise. The terms “a,” “an” and “the” mean “one or more,” unless expressly specified otherwise.

The terms “an embodiment,” “embodiment,” “embodiments,” “the embodiment,” “the embodiments,” “one or more embodiments,” “some embodiments,” and “one embodiment” mean “one or more (but not all) embodiments of the present invention(s),” unless expressly specified otherwise.

For instance, while specific geometrical configurations for the modular generator, such as cylindrical, cubic, and rectangular designs, have been described, it is contemplated that other geometrical shapes and configurations may be employed based on specific application needs or material constraints. Similarly, while particular materials have been mentioned for constructing the reactor components and electrodes, other materials may be equally applicable and are intended to fall within the scope of this disclosure.

The described methods and systems can be implemented in various combinations to achieve desired outcomes. For example, the integration of additional process units or the reconfiguration of existing units can be employed to optimize the system for particular uses, such as increasing hydrogen production, enhancing freshwater recovery, or improving bioelectricity generation. Additionally, the described modular design allows for the selective replacement or upgrading of components, enabling customization and scalability for different applications or operational environments.

Furthermore, the control systems, feedback mechanisms, and other operational aspects of the invention are described with specific examples; however, these should not be interpreted as limiting. Alternative control strategies, monitoring systems, or feedback loops, whether currently known or later developed, can be utilized to achieve similar or improved outcomes. The disclosure is also not limited to the specific electrode configurations or electrode materials described herein, as alternative designs may provide similar benefits and are considered within the scope of this disclosure.

In an embodiment, the present disclosure provides an integrated modular system designed for the concurrent production of natural hydrogen, freshwater, and bioelectricity. The system may integrate multiple processes, such as fermentation, desalination, and electrolysis, within a single modular framework, allowing for the efficient and simultaneous production of these essential resources. The modular design may enable flexibility in configuration, making the system adaptable to a wide range of applications, including large-scale industrial operations and smaller, localized setups.

The system, as described in the present disclosure, may feature various geometrical configurations for the generator, including but not limited to cylindrical, cubic, and rectangular. Other polygonal shapes of the generator may include triangular, square, pentagonal, hexagonal, heptagonal, octagonal, and trapezoidal. Each configuration may be tailored to optimize the system's performance for specific uses. The modularity of the system may allow for the easy replacement, upgrading, or reconfiguration of components, thereby improving the system's scalability and adaptability. This versatility could be particularly advantageous for meeting varying production demands, whether in urban settings or in remote locations where resources may be limited.

In another embodiment, the system may incorporate advanced control mechanisms and feedback loops that monitor and regulate the production processes, ensuring optimal efficiency and output. The control systems may allow for the dynamic adjustment of production ratios between hydrogen, freshwater, and bioelectricity based on real-time requirements. Additionally, the present disclosure may include electrode and membrane designs that improve the efficiency of the electrolysis and fermentation processes, contributing to higher yields of hydrogen and bioelectricity while maintaining the quality of the freshwater output.

Overall, the systems and methods described in the present disclosure may represent an advancement in sustainable technology by offering a comprehensive solution that addresses the growing demand for clean energy, water, and electricity. By combining these processes into a unified system, the present disclosure may maximize resource utilization and minimize environmental impact, providing an efficient, adaptable, and scalable solution for the future of energy and water production.

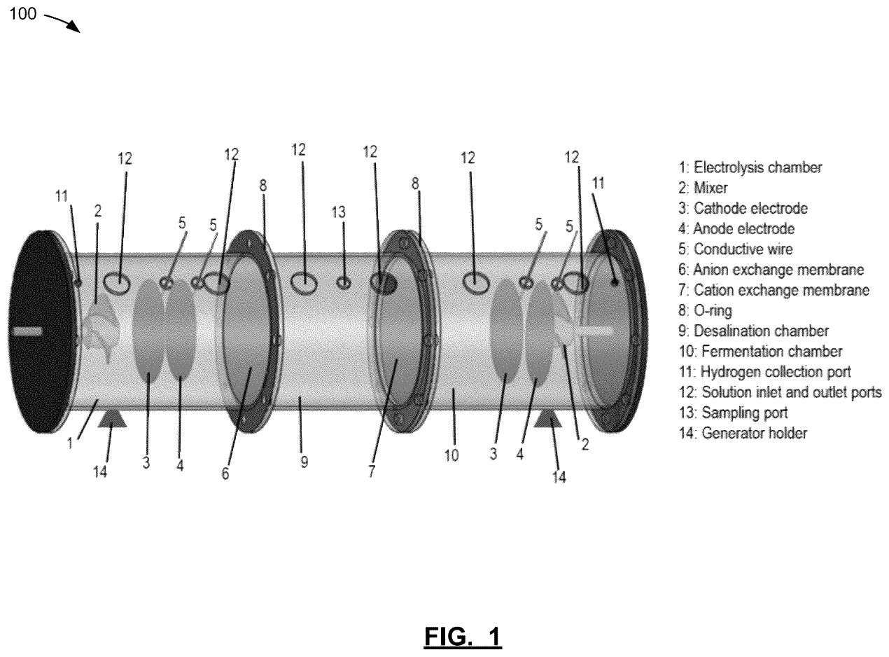

Reference is first made to FIG. 1, which illustrates an embodiment of a modular three-chambered cylindrical natural hydrogen, freshwater, and bioelectricity generator system 100. In this embodiment, the generator system may comprise three distinct compartments dedicated to fermentation, desalination, and electrolysis processes, which function concurrently to produce hydrogen, freshwater, and bioelectricity.

The generator system includes an electrolysis compartment 1. The electrolysis chamber 1 may also be referred to as the electrolysis compartment. The electrolysis chamber 1 may be located on one side of the generator system. The electrolysis chamber 1 may comprise an electrolyte solution. The electrolyte solution may further comprise a saline mixture, including sodium, chlorine ions, and other anions and cations. Within the electrolysis chamber 1, first and second electrodes 3, 4 may be spaced apart and at least partially submerged in the electrolyte solution. Holes may be bored on the electrodes 3, 4, and conductive wires 5 may be wound around them. The electrodes 3, 4 can be fixed in place using the holes located at the top of the electrolysis chamber. The electrodes 3, 4 may be connected to an external electrical circuit through an applied external resistance. The average distance between the anode and cathode electrodes in an embodiment may range from approximately 5 to 10 centimeters. Additionally, inlet and outlet holes 12 may be provided at the top of the electrolysis chamber 1 for the solution to enter and exit the system 100. The hydrogen gas generated during the process may be collected through a hole 11 at the top of the chamber.

The generator system includes a fermentation chamber 10. The fermentation chamber 10 may also be referred to as the electrolysis compartment. The fermentation chamber 10 may be located at the opposite end of the generator system 100. The fermentation chamber 10 may be designed to accommodate a mixture of biomass waste and an anaerobic microorganism solution comprising organic substances and various types of bacteria. The bacteria may include hydrolytic, acidogenic, acetogenic, and methanogenic bacteria, which work in synergy to break down complex organic materials and produce bioenergy, primarily in the form of hydrogen gas. The fermentation chamber 10 may also include its own first and second spaced apart electrodes that are at least partially immersed in the solution, facilitating bioenergy collection. These electrodes may feature bored holes with conductive wires wound around them, and they are fixed in place using the holes at the top of the fermentation chamber. The electrodes may be connected to an external multimeter to measure the potential produced within the chamber. The hydrogen gas produced in this compartment may also be collected from a distinct hole located at the top of the chamber.

The generator system includes a desalination chamber 9. The desalination chamber 9 may also be referred to as the desalination compartment. The desalination chamber 9 may is located between the electrolysis and fermentation chambers. The desalination chamber 9 may be configured to comprise a saline solution comprising sodium, chlorine ions, and other anions and cations. The desalination chamber 9 may be separated from the electrolysis and fermentation chambers by an anion exchange membrane 6 and a cation exchange membrane 7, respectively. The membranes 6, 7, are configured to facilitate ion exchange processes for desalination. To provide a leak-proof operation, the chambers 1, 9, and 10 may be clamped together using gaskets and O-rings 8, secured by stainless steel bolts.

Furthermore, the generator system 100 may include mixers, such as paddle-type, spiral, and ribbon mixers, placed horizontally within the fermentation and electrolysis chambers. The mixers may be provided to maintain a homogeneous solution within the chambers, for overall efficiency and performance of the generator system 100.

In this embodiment, the generator system 100 provides an integrated approach to producing hydrogen, freshwater, and bioelectricity within a modular, cylindrical structure, designed to optimize the concurrent operation of these processes.

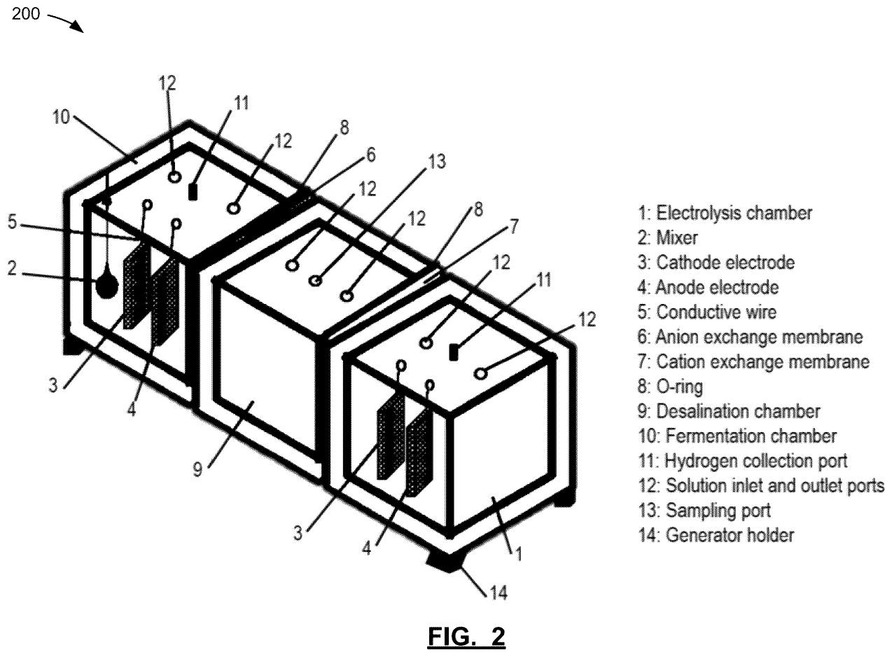

Reference is now made to FIG. 2, which illustrates an embodiment of a modular three-chambered cubic natural hydrogen, freshwater, and bioelectricity generator system 200. In this embodiment, the generator system 200 may comprise three distinct compartments dedicated to fermentation, desalination, and electrolysis processes, which function concurrently to produce hydrogen, freshwater, and bioelectricity.

The generator system 200 includes an electrolysis compartment 1. The electrolysis compartment 1 may also be referred to as the electrolysis chamber. The electrolysis compartment 1 may be located on one side of the generator system 200. The electrolysis compartment 1 may comprise an electrolyte solution. The electrolyte solution may further comprise a saline mixture, including sodium, chlorine ions, and other anions and cations. Within the electrolysis compartment 1, first and second electrodes 3, 4 may be spaced apart and at least partially submerged in the electrolyte solution. Holes may be bored on the electrodes 3, 4, and conductive wires 5 may be wound around them. The electrodes 3, 4 can be fixed in place using the holes located at the top of the electrolysis compartment 21. The electrodes 3, 4 may be connected to an external electrical circuit through an applied external resistance. The average distance between the anode and cathode electrodes in an embodiment may range from approximately 5 to 10 centimeters. Additionally, inlet and outlet holes 12 may be provided at the top of the electrolysis compartment 1 for the solution to enter and exit the system 200. The hydrogen gas generated during the process may be collected through a hole 11 at the top of the compartment.

The generator system 200 includes a fermentation compartment 10. The fermentation compartment 10 may also be referred to as the fermentation chamber. The fermentation compartment 10 may be located at the opposite end of the generator system 200. The fermentation compartment 10 may be designed to accommodate a mixture of biomass waste and an anaerobic microorganism solution comprising organic substances and various types of bacteria. The bacteria may include hydrolytic, acidogenic, acetogenic, and methanogenic bacteria, which work in synergy to break down complex organic materials and produce bioenergy, primarily in the form of hydrogen gas. The fermentation compartment 10 may also include its own first and second spaced apart electrodes that are at least partially immersed in the solution, facilitating bioenergy collection. The electrodes may feature bored holes with conductive wires wound around them. The electrodes are fixed in place using the holes at the top of the fermentation compartment 10. The electrodes may be connected to an external multimeter to measure the potential produced within the compartment. The hydrogen gas produced in this compartment may also be collected from a distinct hole located at the top of the compartment.

The generator system 200 includes a desalination compartment 9. The desalination compartment 9 may also be referred to as the desalination chamber. The desalination compartment 9 may be located between the electrolysis and fermentation compartments. The desalination compartment 9 may be configured to comprise a saline solution comprising sodium, chlorine ions, and other anions and cations. The desalination compartment 9 may be separated from the electrolysis and fermentation compartments by an anion exchange membrane 6 and a cation exchange membrane 7, respectively. The membranes 6, 7 are configured to facilitate ion exchange processes for desalination. To provide a leak-proof operation, the compartments 1, 9, and 10 may be clamped together using gaskets and O-rings 8, secured by stainless steel bolts.

Furthermore, the generator system 200 may include mixers, such as paddle-type, spiral, and ribbon mixers, placed horizontally within the fermentation and electrolysis compartments. The mixers may be provided to maintain a homogeneous solution within the compartments, enhancing the overall efficiency and performance of the generator system 200.

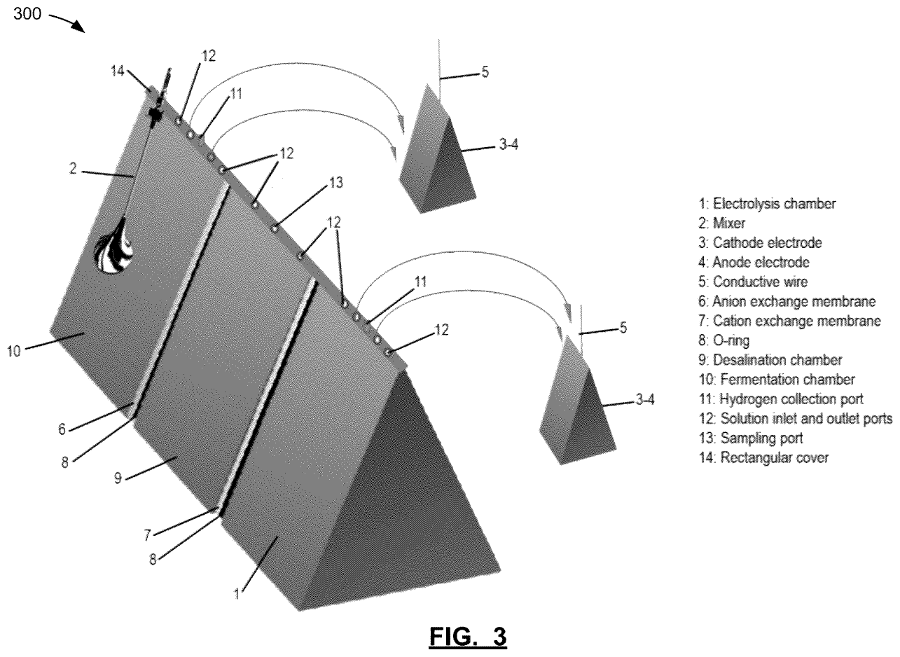

Reference is now made to FIG. 3, which illustrates an embodiment of a modular three-chambered triangular natural hydrogen, freshwater, and bioelectricity generator system 300. In this embodiment, the generator system 300 comprises fermentation, desalination, and electrolysis compartments arranged in a triangular configuration. The triangular design may provide structural advantages and facilitate the integration of the compartments within compact spaces. The generator system 300 includes an external square-shaped cover with holes on the upper part of the generator, specifically designed and built to facilitate the collection and injection of samples.

In an embodiment, each compartment in the generator system 300 may function similarly to the corresponding compartments described in FIG. 1, with similar features including electrodes, membranes, and mixers applied to optimize the concurrent production of hydrogen, freshwater, and bioelectricity. The features, configurations, and components described in FIG. 1 may also be adapted and utilized in this triangular configuration, ensuring consistent performance and efficiency across different structural embodiments.

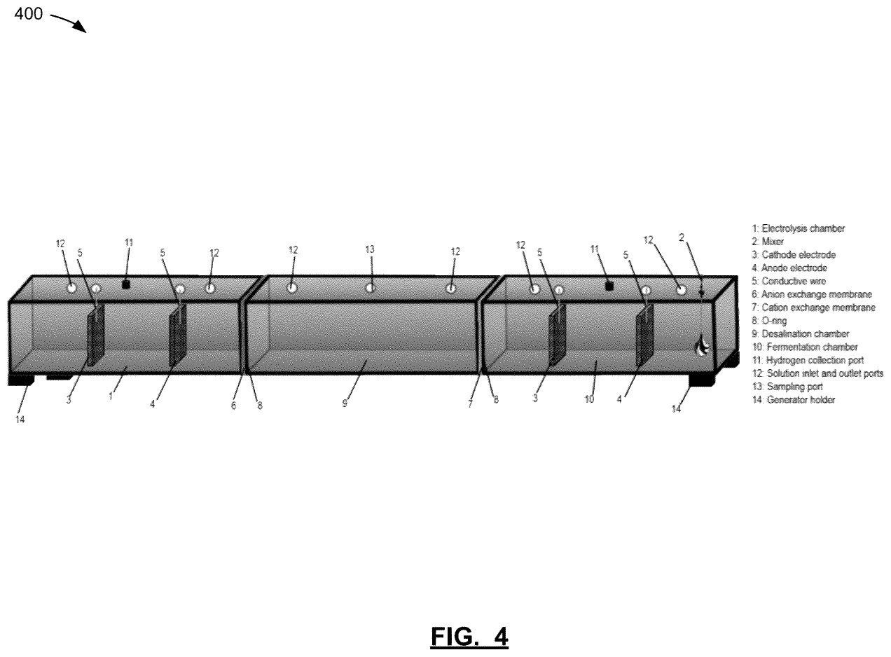

Reference is now made to FIG. 4, which illustrates an embodiment of a modular three-chambered rectangular natural hydrogen, freshwater, and bioelectricity generator system 400. In this embodiment, the generator system 400 comprises fermentation, desalination, and electrolysis compartments arranged in a rectangular configuration. The rectangular design may provide a streamlined structure that can be easily integrated into various settings. Each compartment in this system 400 may function similarly to the corresponding compartments described in FIG. 1, with features such as electrodes, membranes, and mixers applied to optimize the concurrent production of hydrogen, freshwater, and bioelectricity. The features, configurations, and components described in FIG. 1 may also be adapted and utilized in this rectangular configuration to ensure consistent performance and operational efficiency across different structural embodiments.

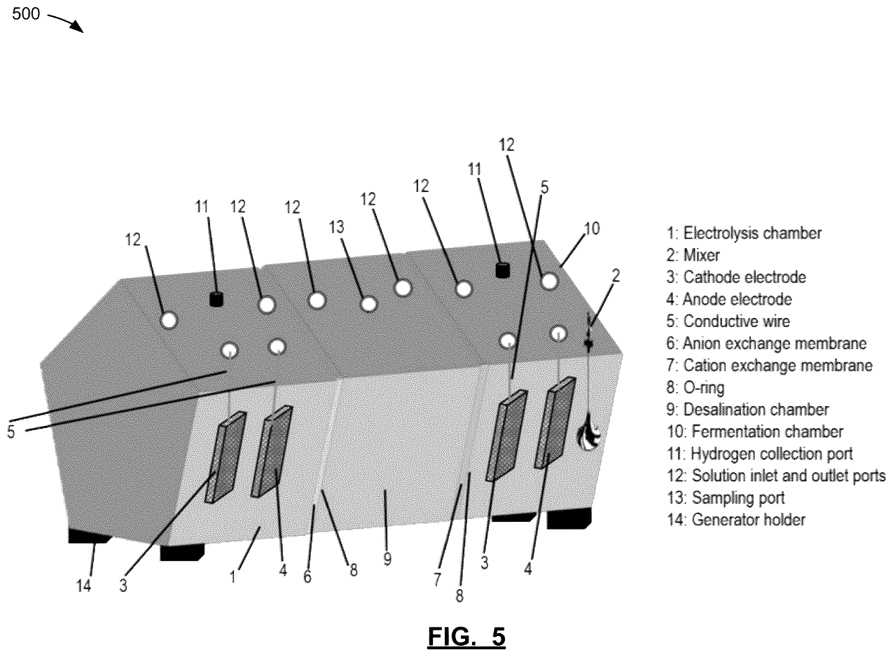

Reference is now made to FIG. 5, which illustrates an embodiment of a modular three-chambered pentagonal natural hydrogen, freshwater, and bioelectricity generator system 500. In this embodiment, the generator system 500 comprises fermentation, desalination, and electrolysis compartments arranged in a pentagonal configuration. The pentagonal design may offer unique spatial advantages, potentially enhancing the distribution of components and optimizing the system's integration into specific environments. Each compartment in this system 500 may function similarly to the corresponding compartments described in FIG. 1, with features such as electrodes, membranes, and mixers applied to optimize the concurrent production of hydrogen, freshwater, and bioelectricity. The features, configurations, and components described in FIG. 1 may also be adapted and utilized in this pentagonal configuration, ensuring consistent performance and efficiency across different structural embodiments.

The generator or reactor system, as described in the present embodiments, can be constructed using various types of materials selected based on the specific application requirements, environmental conditions, and desired durability. These materials may include, but are not limited to, stainless steel, carbon steel, and glass-lined steel, which offer robust structural integrity and corrosion resistance. Additionally, nickel alloys such as hastelloy and monel, as well as titanium, may be utilized for their superior resistance to chemical corrosion and high-temperature stability. In certain embodiments, the reactor may be constructed using reinforced concrete or inconel (a nickel-chromium alloy) for improved strength and durability in extreme conditions. The use of polymers, including polytetrafluoroethylene (PTFE), polyvinylidene fluoride (PVDF), polyethylene (PE), and polypropylene (PP), may be considered for applications requiring lightweight, corrosion-resistant materials. Furthermore, borosilicate glass may be employed in scenarios where chemical resistance and transparency are advantageous for monitoring processes within the reactor system.

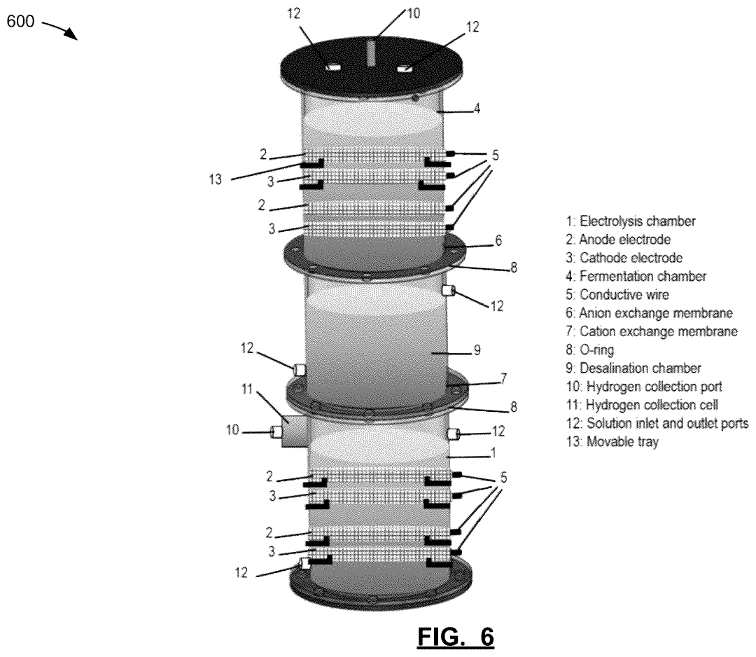

Reference is now made to FIG. 6, which illustrates an embodiment of an integrated modular cascaded generator system 600 for producing concurrent natural hydrogen, freshwater, and bioelectricity. In this embodiment, the generator system 600 may comprise three distinct compartments dedicated to fermentation, desalination, and electrolysis processes, which are arranged in a cascaded configuration to optimize the concurrent production of hydrogen, freshwater, and bioelectricity.

The generator system 600 includes an electrolysis compartment 1. The electrolysis compartment 1 may be located at the bottom of the generator system 600. The electrolysis compartment 1 may comprise an electrolyte solution, which further comprises a saline mixture, including sodium, chlorine ions, and other anions and cations. Within the electrolysis compartment 1, first and second electrodes 2, 3 may be spaced apart and placed parallel to each other on movable rails within the system. Holes may be bored on the electrodes 2, 3, and conductive wires 5 may be wound around them. The electrodes 2, 3 can be fixed in place using the holes located on the movable rails. The electrodes 2, 3 may be connected to an external electrical circuit through an applied external resistance. The average distance between the anode and cathode electrodes may range from approximately 5 to 10 centimeters. Additionally, multiple electrodes may be placed in the chamber to improve the system's performance. An input may be provided at the top right of the electrolysis compartment 1 for solution feeding, and an output may be located at the bottom for solution collection. Hydrogen gas generated during the process may be collected in an external collection cell 11, with the hydrogen gas being collected through a hole 10 on the left side of the external collection cell 11.

The generator system 600 also includes a fermentation compartment 4. The fermentation compartment 4 may be located on the opposite end to the electrolysis chamber. In an embodiment, the fermentation compartment 4 may be located above the desalination compartment 9. The fermentation compartment 4 may be designed to accommodate a mixture of biomass waste and an anaerobic microorganism solution comprising organic substances and various types of bacteria. The bacteria may include hydrolytic, acidogenic, acetogenic, and methanogenic bacteria, which work in synergy to break down complex organic materials and produce bioenergy, primarily in the form of hydrogen gas. Similar to the electrolysis compartment, separate first and second electrodes 2, 3 may be spaced apart and placed parallel to each other on movable rails. The electrodes 2, 3 may feature bored holes with conductive wires wound around them. The electrodes 2, 3 may be connected to an external multimeter to measure the potential produced within the compartment. The average distance between the anode and cathode electrodes may range from approximately 5 to 10 centimeters. Multiple electrodes may be placed in the fermentation compartment 70 to improve performance. The hydrogen gas produced in the fermentation compartment may also be collected through a hole placed at the top of the compartment.

The generator system 600 further includes a desalination compartment 9. The desalination compartment 9 may be located between the electrolysis and fermentation compartments. The desalination compartment 9 may be configured to comprise a saline solution, including sodium, chlorine ions, and other anions and cations. Anion and cation exchange membranes 6, 7 may be placed at the top and bottom of the desalination compartment 9, respectively, to separate it from the electrolysis and fermentation compartments and to facilitate ion exchange processes for desalination. An input may be provided at the top of the desalination compartment 9 for solution feeding, and an output may be located at the bottom for solution collection.

To provide a leak-proof operation, the compartments 1, 4, and 9 may be clamped together using gaskets and O-rings 8, secured by stainless steel bolts.

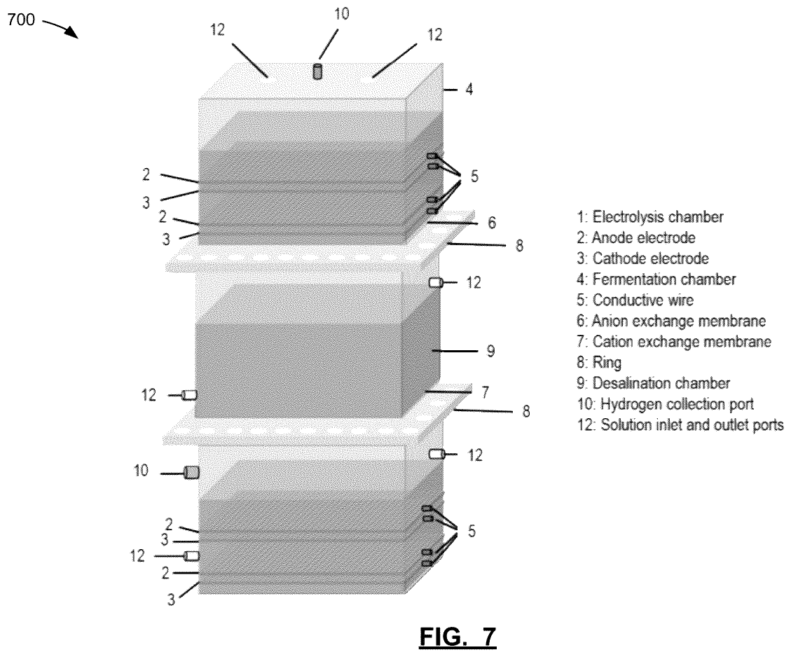

Reference is now made to FIG. 7, which illustrates an embodiment of a cascaded three-chambered cubic natural hydrogen, freshwater, and bioelectricity generator system 700. In this embodiment, the generator system 700 is designed with a cubic structure to produce hydrogen, freshwater, and bioelectricity concurrently. The generator system 700 comprises fermentation, desalination, and electrolysis compartments, which are arranged in a cascaded configuration. The compartments and components of the system 700 may function similarly to those described in FIG. 6, with appropriate adjustments made to accommodate the cubic configuration. These adjustments may include modifications in the layout and arrangement of electrodes, membranes, and other internal components to optimize the system's performance within the cubic design.

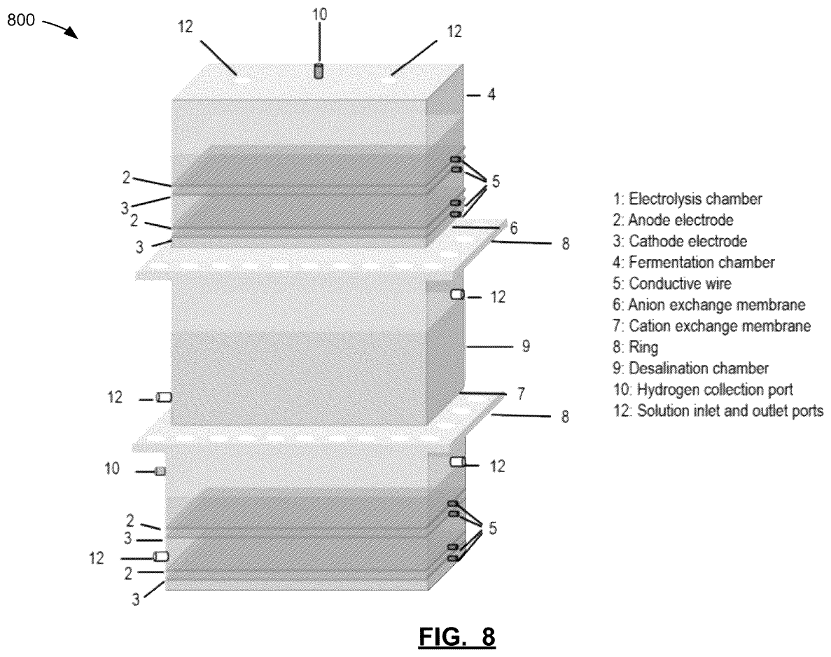

Reference is now made to FIG. 8, which illustrates an embodiment of a cascaded three-chambered square natural hydrogen, freshwater, and bioelectricity generator system 800. In this embodiment, the generator system 800 is designed with a square structure to produce hydrogen, freshwater, and bioelectricity concurrently. The generator system 800 comprises fermentation, desalination, and electrolysis compartments, which are arranged in a cascaded configuration. The compartments and components of the system 800 may function similarly to those described in FIG. 7, with appropriate adjustments made to accommodate the square configuration. These adjustments may include modifications in the layout and arrangement of electrodes, membranes, and other internal components to optimize the system's performance within the square design.

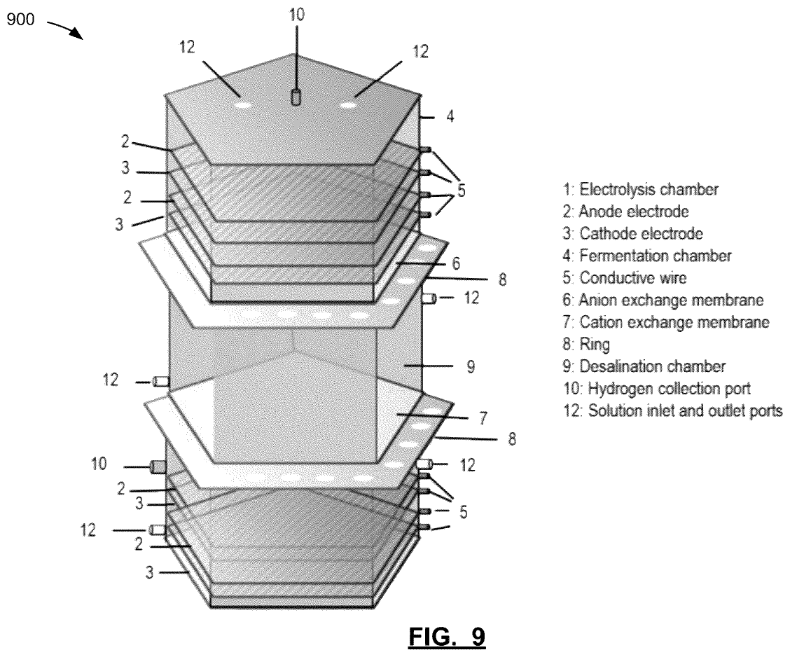

Reference is now made to FIG. 9, which illustrates an embodiment of a cascaded three-chambered pentagonal natural hydrogen, freshwater, and bioelectricity generator system 900. In this embodiment, the generator system 900 is designed with a pentagonal structure to produce hydrogen, freshwater, and bioelectricity concurrently. The generator system 900 comprises fermentation, desalination, and electrolysis compartments, which are arranged in a cascaded configuration. The compartments and components of the system 900 may function similarly to those described in FIG. 7, with appropriate adjustments made to accommodate the pentagonal configuration. These adjustments may include modifications in the layout and arrangement of electrodes, membranes, and other internal components to optimize the system's performance within the pentagonal design.

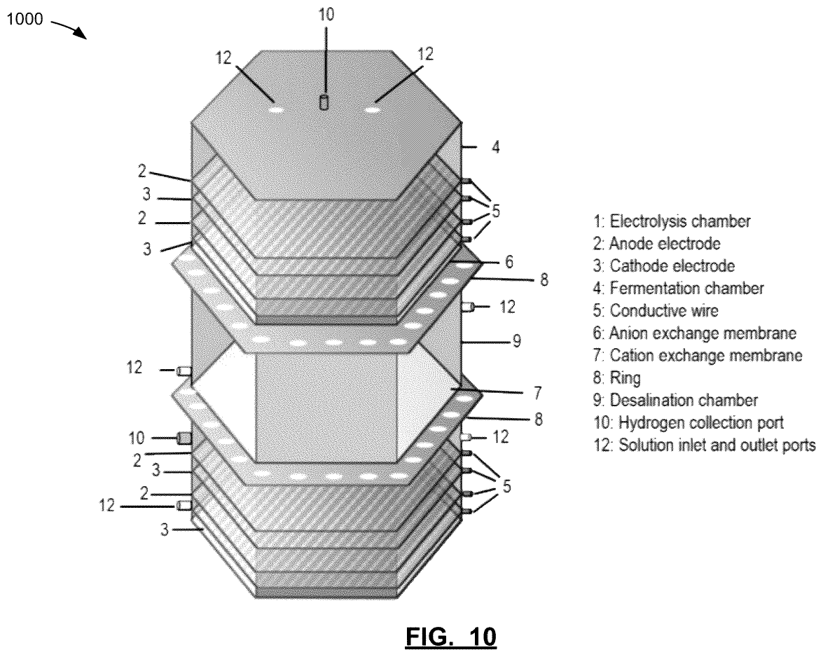

Reference is now made to FIG. 10, which illustrates an embodiment of a cascaded three-chambered hexagonal natural hydrogen, freshwater, and bioelectricity generator system 1000. In this embodiment, the generator system 1000 is designed with a hexagonal structure to produce hydrogen, freshwater, and bioelectricity concurrently. The generator system 1000 comprises fermentation, desalination, and electrolysis compartments, which are arranged in a cascaded configuration. The compartments and components of the system 1000 may function similarly to those described in FIG. 7, with appropriate adjustments made to accommodate the hexagonal configuration. These adjustments may include modifications in the layout and arrangement of electrodes, membranes, and other internal components to optimize the system's performance within the hexagonal design.

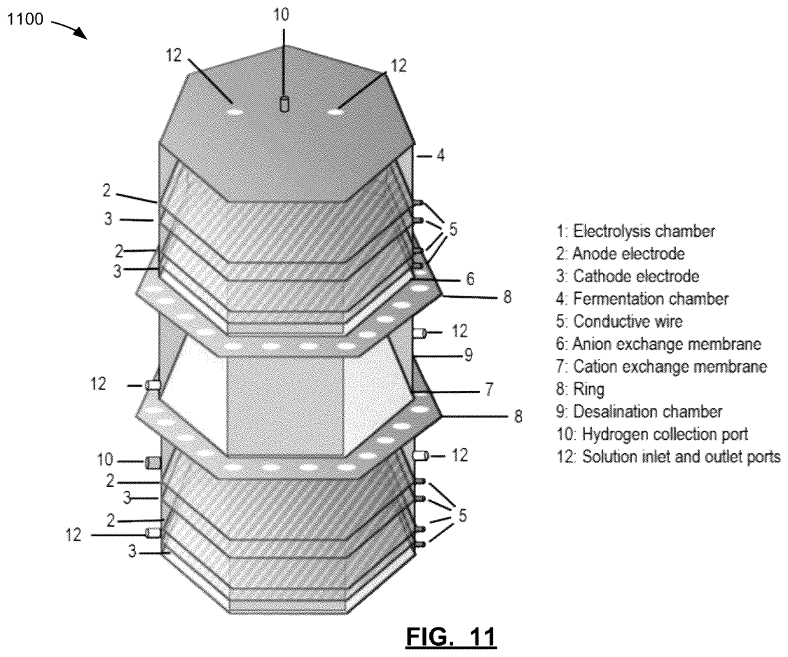

Reference is now made to FIG. 11, which illustrates an embodiment of a cascaded three-chambered heptagonal natural hydrogen, freshwater, and bioelectricity generator system 1100. In this embodiment, the generator system 1100 is designed with a heptagonal structure to produce hydrogen, freshwater, and bioelectricity concurrently. The generator system 1100 comprises fermentation, desalination, and electrolysis compartments, which are arranged in a cascaded configuration. The compartments and components of the system 1100 may function similarly to those described in FIG. 7, with appropriate adjustments made to accommodate the heptagonal configuration. These adjustments may include modifications in the layout and arrangement of electrodes, membranes, and other internal components to optimize the system's performance within the heptagonal design.

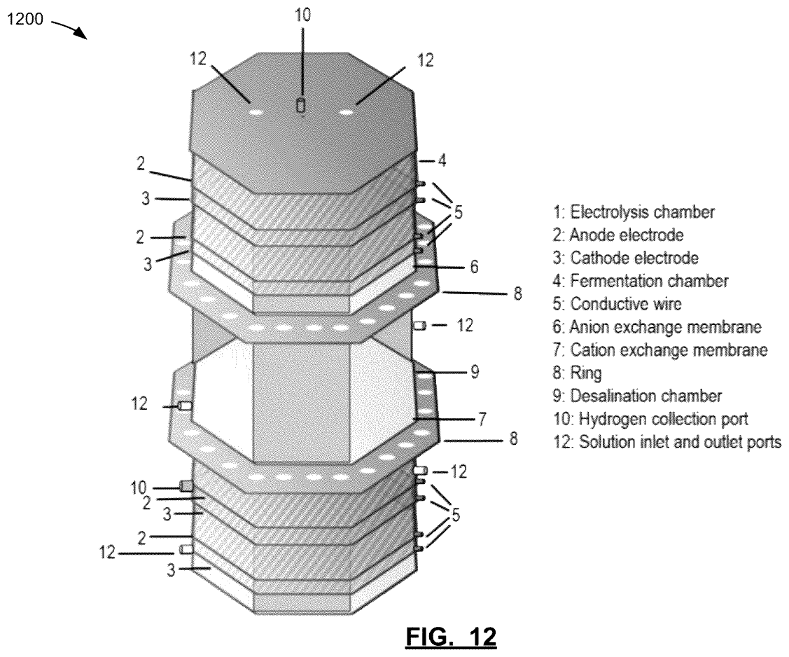

Reference is now made to FIG. 12, which illustrates an embodiment of a cascaded three-chambered octagonal natural hydrogen, freshwater, and bioelectricity generator system 1200. In this embodiment, the generator system 1200 is designed with an octagonal structure to produce hydrogen, freshwater, and bioelectricity concurrently. The generator system 1200 comprises fermentation, desalination, and electrolysis compartments, which are arranged in a cascaded configuration. The compartments and components of the system 1200 may function similarly to those described in FIG. 7, with appropriate adjustments made to accommodate the octagonal configuration. These adjustments may include modifications in the layout and arrangement of electrodes, membranes, and other internal components to optimize the system's performance within the octagonal design.

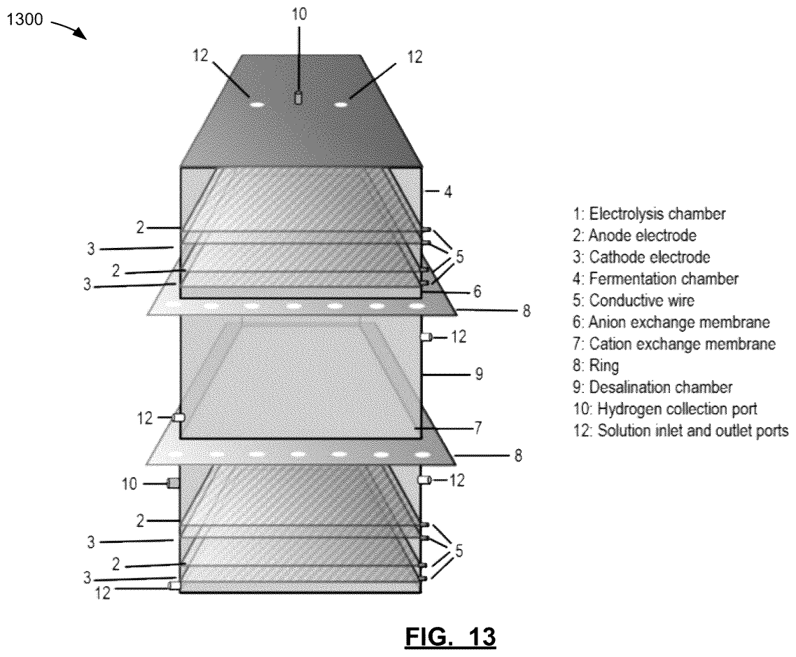

Reference is now made to FIG. 13, which illustrates an embodiment of a cascaded three-chambered rectangular natural hydrogen, freshwater, and bioelectricity generator system 1300. In this embodiment, the generator system 1300 is designed with a rectangular structure to produce hydrogen, freshwater, and bioelectricity concurrently. The generator system 1300 comprises fermentation, desalination, and electrolysis compartments, which are arranged in a cascaded configuration. The compartments and components of the system 1300 may function similarly to those described in FIG. 7, with appropriate adjustments made to accommodate the rectangular configuration. These adjustments may include modifications in the layout and arrangement of electrodes, membranes, and other internal components to optimize the system's performance within the rectangular design.

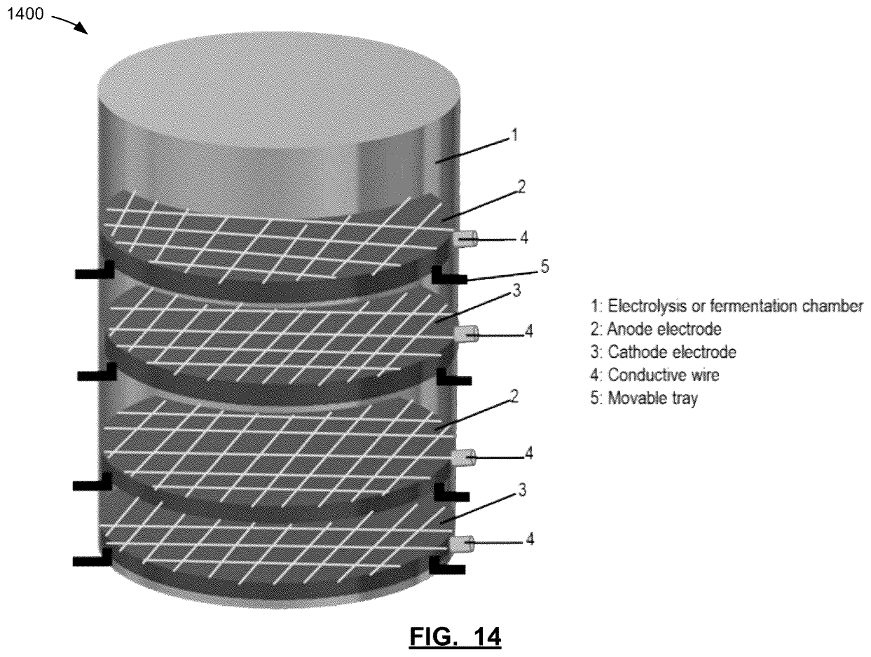

Reference is now made to FIG. 14, which illustrates an embodiment of a reactor system 1400 operated with cascaded electrodes. In this embodiment, the reactor system 1400 may be designed by integrating multiple movable trays on the walls of the reactor to facilitate the placement of electrodes 2, 3 within the system 1400 in a cascaded arrangement. The movable trays may allow for the adjustment of the number and positioning of the electrodes, depending on the specific application and operational requirements of the reactor system 1400. These trays can be repositioned or reconfigured to optimize contact between the electrodes and the reactive solution.

The movable tray design provides flexibility in electrode configuration, allowing the reactor system 1400 to be adapted for various purposes, such as improving electrochemical reactions or optimizing energy production. The inner design described in FIG. 14 may be applicable to reactors with different geometrical shapes, including but not limited to cubic, rectangular, and square configurations. Furthermore, the movable trays may be easily integrated into reactors of more complex geometrical shapes, such as pentagonal, hexagonal, heptagonal, octagonal, and trapezoidal, for electrode placement across a range of reactor designs.

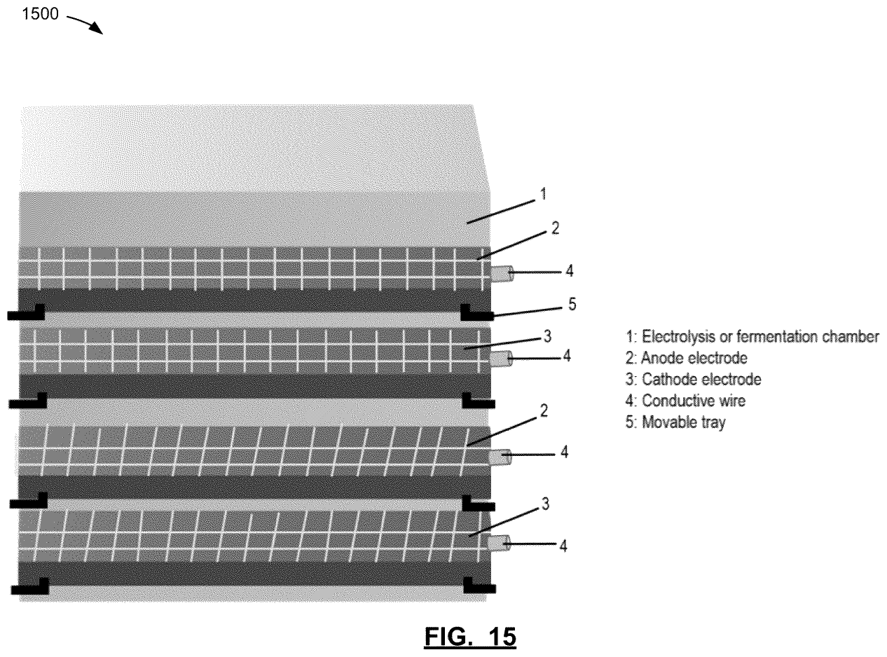

Reference is now made to FIG. 15, which illustrates an embodiment of electrode placement within cubic, square, and rectangular-shaped reactor systems. In this embodiment, the electrodes may be arranged on movable trays within the electrolysis and fermentation chambers. The design allows for flexibility in electrode positioning and number, similar to the operation described in FIG. 14. The electrodes can be adjusted to optimize the interaction with the reactive solution, enhancing the overall efficiency of the electrochemical and biological processes within the reactor systems.

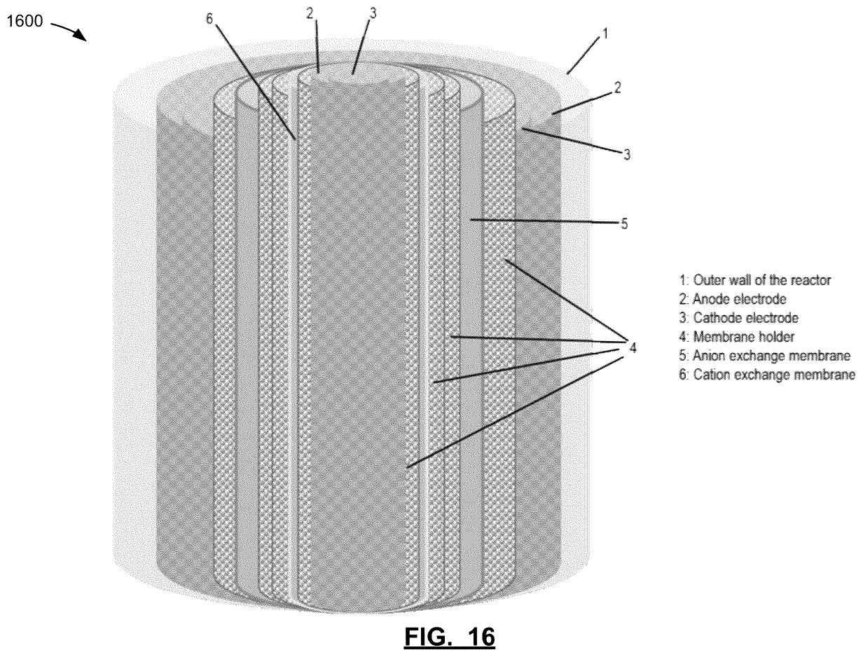

Reference is now made to FIG. 16, which illustrates an embodiment of an integrated modular intertwined generator system 1600. In this embodiment, the generator system 1700 may be designed with intertwined compartments for the concurrent production of natural hydrogen, freshwater, and bioelectricity. The generator system 1600 comprises three interconnected chambers: fermentation, electrolysis, and desalination.

The generator system 1600 includes an electrolysis chamber, which may be located in the interior part of the generator system 1600. The electrolysis chamber may include cylindrical electrodes 2, 3. The electrodes 2, 3 may be separated from the desalination chamber by a cation exchange membrane. Holes may be bored into the electrodes, and conductive wires may be wound around the electrodes. The electrodes 2, 3 may be fixed in place using the holes located at the top of the electrolysis chamber. The electrodes 2, 3 may be connected to an external electrical circuit via an applied external resistance. In an embodiment, the average distance between the anode and cathode electrodes may range from approximately 5 to 10 centimeters. Additionally, inlet and outlet holes may be provided at the top of the electrolysis chamber for the solution to enter and exit the system 1600. The hydrogen gas generated during the process may be collected through a hole at the top of the electrolysis chamber.

The generator system 1600 further includes a desalination chamber, which may be positioned in the middle of the generator system 1600, between the electrolysis and fermentation chambers. The desalination chamber may be separated from the outermost fermentation chamber by an anion exchange membrane 5. The desalination chamber may include input and output ports at the top for solution feeding and collection.

The generator system 1600 further includes a fermentation chamber located at the outermost part of the generator system 1600. The fermentation chamber may include its own first and second spaced apart electrodes 2, 3 that are placed in the solution to facilitate bioenergy collection. Holes may be bored into the electrodes, and conductive wires may be wound around the electrodes. The electrodes may be fixed in place using the holes at the top of the fermentation chamber. The electrodes may be connected to an external multimeter to measure the potential produced within the chamber. The hydrogen gas produced in the fermentation chamber may be collected through a hole at the top of the chamber.

The modular generator system 1600 may be constructed in various geometrical shapes, including but not limited to cylindrical, cubic, triangular, rectangular, square, pentagonal, hexagonal, heptagonal, octagonal, and trapezoidal. The reactor chambers within the system 1600 may be constructed using various materials such as stainless steel, carbon steel, glass-lined steel, nickel alloys (e.g., hastelloy, monel), titanium, reinforced concrete, inconel (nickel-chromium alloy), polymers (e.g., polytetrafluoroethylene (PTFE), polyvinylidene fluoride (PVDF), polyethylene (PE), polypropylene (PP)), and borosilicate glass.

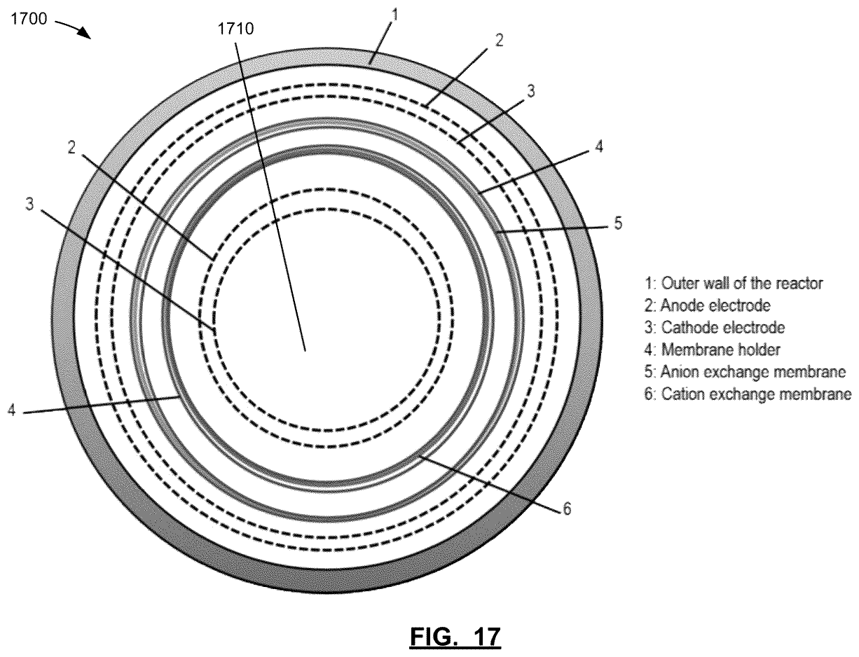

Reference is now made to FIG. 17, which illustrates an embodiment of the cross-section view of an intertwined natural hydrogen, freshwater, and bioelectricity generator system 1700. In the embodiment, the generator system is designed with a concentric arrangement of its three primary chambers: electrolysis, desalination, and fermentation.

The electrolysis chamber 1710 is centrally located in the middle of the generator system 1700. Surrounding the electrolysis chamber is the desalination chamber, which is positioned concentrically around the electrolysis chamber 1710. The outermost layer of the generator system 1700 is the fermentation chamber, which encases both the desalination and electrolysis chambers. This concentric design facilitates efficient interaction between the chambers, allowing the generator system 1700 to simultaneously produce hydrogen, freshwater, and bioelectricity. The arrangement ensures that the processes within each chamber are effectively isolated yet closely integrated, optimizing the overall performance of the generator system 1700.

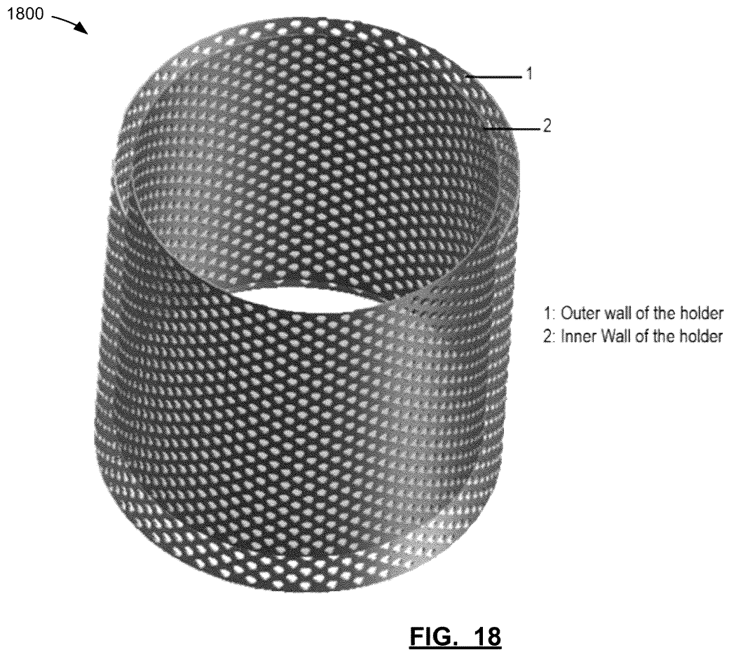

Reference is now made to FIG. 18, which illustrates an embodiment of a membrane holder 1800 used within the generator system. In this embodiment, the membrane holder 1800 is designed as a cylindrical structure with numerous perforations distributed evenly across its surface. These perforations allow for the effective flow of liquids and gases through the membrane, ensuring efficient interaction with the surrounding reactive components within the generator system. The membrane holder 1800 may be used to securely position and support ion exchange membranes or other types of membranes within the system, maintaining their stability and enhancing the overall performance of processes such as electrolysis, desalination, and fermentation.

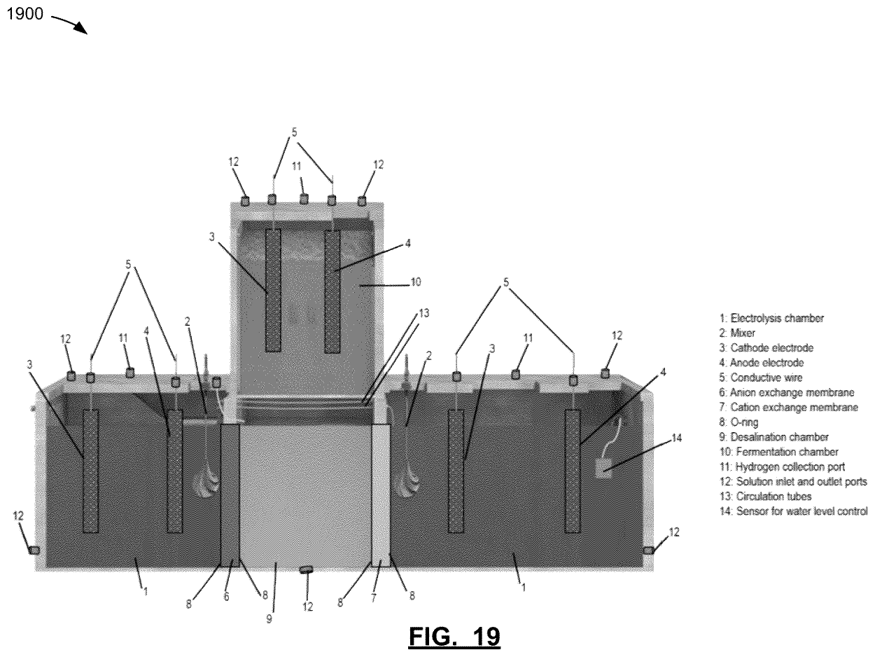

Reference is now made to FIG. 19, which illustrates an embodiment of a multi-chamber generator system 1900 designed for the concurrent production of natural hydrogen, desalination, and bioenergy. In this embodiment, the generator system 1900 may include four interconnected chambers: one fermentation chamber 10, two electrolysis chambers 1 (on the sides), and one desalination chamber 9.

In an embodiment, the fermentation chamber 10 is located at the top of the generator system 1900. In the fermentation chamber, first and second spaced-apart electrodes 3, 4 are placed in a solution for bioenergy collection. Holes may be bored into the electrodes, and conductive wires 5 may be wound around them. The electrodes 3, 4 may be fixed in place using the holes at the top of the fermentation chamber 10. The electrodes 3, 4 may be connected to an external multimeter to measure the potential produced within the chamber. Hydrogen gas produced in the fermentation chamber 10 may be collected through a hole at the top of the chamber. The fermentation chamber 10 may also include input and output ports for solution feeding and collection.

In an embodiment, the desalination chamber 9 is located below the fermentation chamber 10. The desalination chamber 9 may be separated from the adjacent electrolysis chambers by anion and cation exchange membranes 6, 7, respectively. The desalination chamber 9 may be connected to the electrolysis chambers through a solution inlet, facilitating the movement of liquids between the chambers. A hole may be provided at the bottom of the desalination chamber for the collection of treated water.

In an embodiment, the electrolysis chambers are positioned on either side of the desalination chamber 9. Each electrolysis chamber may include first and second spaced-apart electrodes 3, 4, which are partially placed in the electrolysis chamber's solution. Holes may be bored into the electrodes, and conductive wires may be wound around them. The electrodes 3, 4, are fixed in place using holes at the top of the electrolysis chambers and are connected to an external electrical circuit via an applied external resistance. The average distance between the anode and cathode electrodes may range from approximately 5 to 10 centimeters. Each electrolysis chamber may also include inlet holes at the top and outlet holes at the bottom for the solution to enter and exit the system 1900. Hydrogen gas produced in the electrolysis chambers may be collected through holes at the top of each chamber.

To ensure a leak-proof operation, the electrolysis, fermentation, and desalination chambers may be clamped together using O-rings and bolts. Mixers, such as paddle-type, spiral, and ribbon mixers, may be placed in the electrolysis chambers to maintain a homogeneous solution. Additionally, continuous liquid circulation between the electrolysis chambers may be provided using a circulation tube with pumps. The water level in the desalination and electrolysis chambers may be controlled by placing a water level sensor into one of the electrolysis chambers.

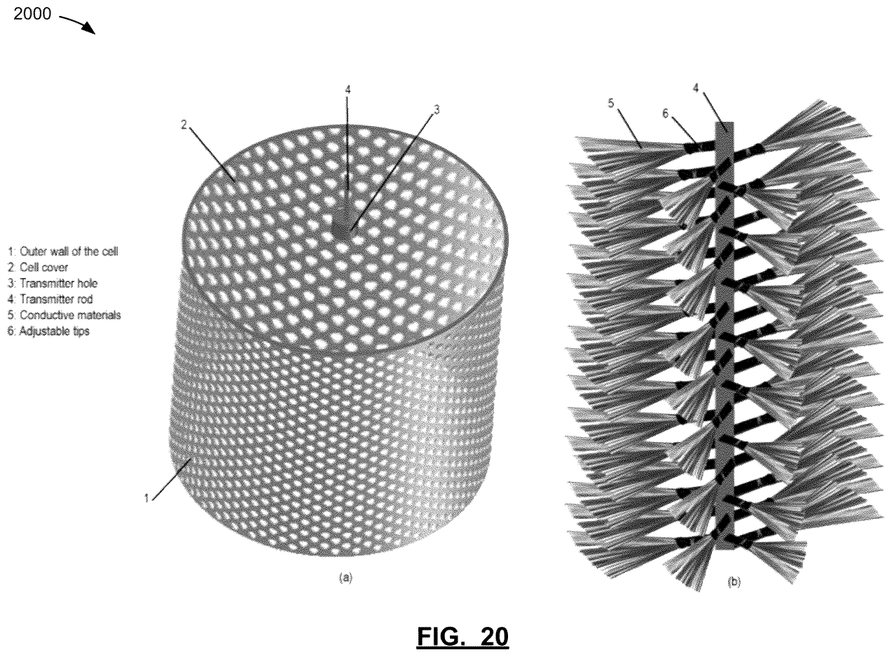

Reference is now made to FIG. 20A and FIG. 20B, which illustrates an embodiment of a modular single cell 2000 designed for electrode placement within a generator system. In this embodiment, the modular cell 2000 may be configured to hold multiple electrodes. The electrodes may be provided in various shapes and configurations to suit different operational needs. The cell 2000 is designed with a non-conductive material-based frame that includes strategically placed holes to facilitate effective liquid flow throughout the system.

In an embodiment, the cylindrical type electrode cell 2000 is designed to accommodate particle and/or scrap-type electrodes. The modular design of the electrode cell 2000 allows it to be adapted for use in different geometric shapes, such as cubes, prisms, or spheres, depending on the specific hydrogen generator type. The holes in the electrode cell can also be configured in different geometric shapes, including square, rectangular, or round, to optimize liquid flow distribution within the cell and the generator.

The modular electrode cell 2000 may further include a conductive rod (FIG. 20B). The rod is used to transfer electricity throughout the electrodes within the cell. The conductive rod may include adjustable tips that allow for the placement of conductive materials of various shapes, providing efficient electricity transfer throughout the electrodes in the electrode cell 2000.

The electrode holder and frame may be constructed from a variety of materials, including stainless steel, glass-lined steel, reinforced concrete, or polymers such as polytetrafluoroethylene (PTFE), polyvinylidene fluoride (PVDF), polyethylene (PE), or polypropylene (PP). The cell 2000 can be designed in various shapes, such as cylindrical, cubic, triangular, rectangular, square, pentagonal, hexagonal, heptagonal, octagonal, or trapezoidal, to suit different generator designs. The conductive rod and electrodes may be made from materials such as carbon, copper, chromium, cadmium, bronze, germanium, graphite, gold, nickel, zinc, lead, platinum, titanium, rhodium, palladium, iridium, ruthenium, and silver, which can be shaped into plates, brushes, rods, and other configurations as needed.

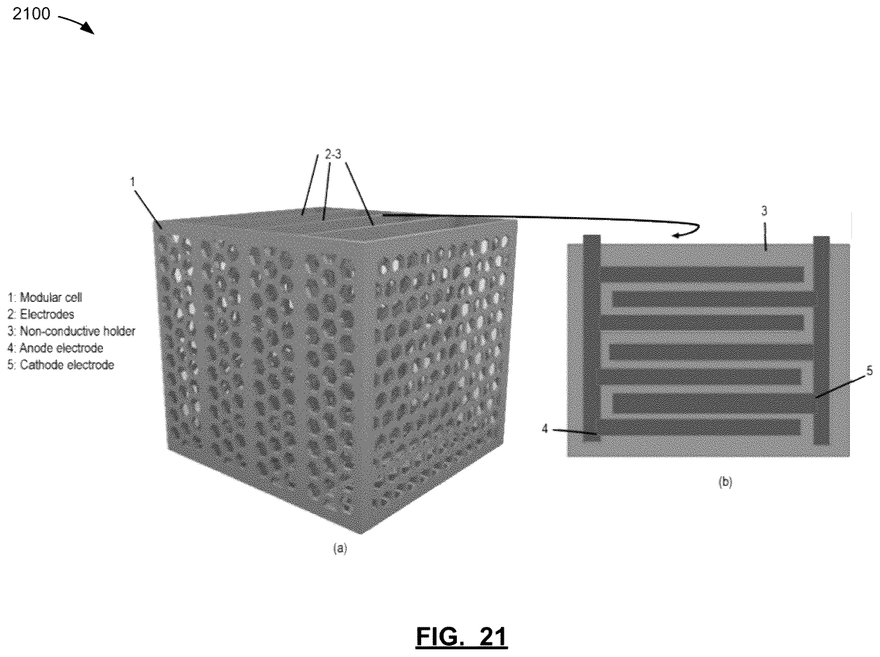

Reference is now made to FIG. 21A and FIG. 21B, which illustrate an embodiment of a modular cascaded electrode cell 2100 designed for enhanced hydrogen production. In this embodiment, the electrode cell 2100 is constructed to facilitate efficient liquid flow and optimize the electrochemical reactions necessary for hydrogen production.

The electrode cell 2100 may include a cubic-type structure, as shown in FIG. 21A. The cell 2100 is designed with multiple holes distributed across its surface to ensure effective liquid flow throughout the cell. The modular design allows for the utilization of plate-type electrodes, as illustrated in FIG. 21B. The non-conductive material-based frame of the cell 2100 holds the electrodes in place, maintaining their stability and positioning within the hydrogen generator system.

The modular electrode cell 2100 can be adapted and used in different geometric shapes, including but not limited to cubes, prisms, and spheres, depending on the specific requirements of the hydrogen generator type. The holes on the electrode cell 2100 can be designed in various geometric shapes, such as square, rectangular, or round, to provide the most appropriate liquid flow distribution within the cell and the overall generator system.

The electrode cell and its holder may be constructed from a variety of materials, including stainless steel, glass-lined steel, reinforced concrete, or polymers such as polytetrafluoroethylene (PTFE), polyvinylidene fluoride (PVDF), polyethylene (PE), or polypropylene (PP). Additionally, the electrode cell can be designed in various shapes, including cylindrical, cubic, triangular, rectangular, square, pentagonal, hexagonal, heptagonal, octagonal, and trapezoidal, to suit different generator designs. The electrodes within the cell may be made from materials such as carbon, copper, chromium, cadmium, bronze, germanium, graphite, gold, nickel, zinc, lead, platinum, titanium, rhodium, palladium, iridium, ruthenium, and silver. The materials can be shaped into plates, rods, or other configurations as needed for the specific electrochemical processes involved in hydrogen production.

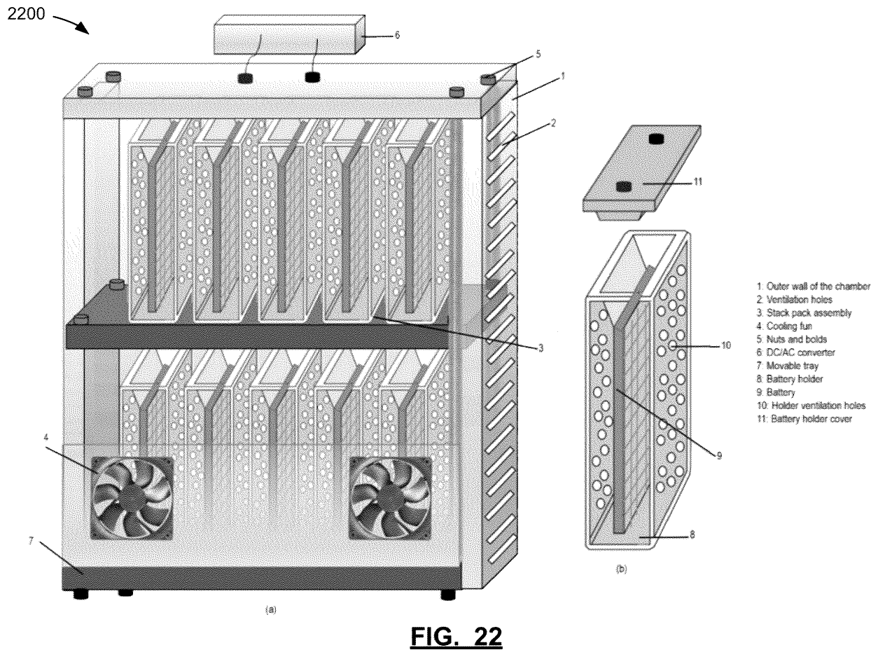

Reference is now made to FIG. 22A and FIG. 22B, which illustrate an embodiment of a modular cascaded bioenergy collector 2200 for the storage of bioenergy produced in the fermentation chamber. In this embodiment, the bioenergy collector 2200 is configured to hold multiple batteries and supercapacitors in various shapes and configurations, utilizing a cascaded design to enhance its energy storage capabilities.

The bioenergy collector 2200 may facilitate energy transfer from the source to the batteries and/or supercapacitors using a DC/AC converter 6, which includes positive and negative connection ports. The side walls of the collector 2200 are designed with holes to provide effective natural airflow, preventing excess heating of the components. The holes can be configured in different geometric shapes, such as square, rectangular, or round, to provide the most appropriate airflow distribution within the collector 2200.

In an embodiment, the collector 2200 is placed on a movable rail or tray 7, which allows for effective natural ventilation. By moving this rail left and right, the efficiency of natural ventilation can be further increased. Additionally, the collector 2200 may be equipped with ventilation fans 4, which may be activated if additional ventilation is required to maintain optimal operating conditions.

The modular energy collector 2200 can be designed in various geometric shapes, including cubes, prisms, or spheres, depending on the type of batteries and supercapacitors being used. The rectangular-type battery and supercapacitor cell, as depicted in FIG. 22B, is configured to hold these components. The cell may include a non-conductive material-based frame with holes to securely hold the batteries and supercapacitors while also providing natural ventilation.

The holes on the battery holder in FIG. 22B can be designed in different geometric shapes, such as square, rectangular, or round, to optimize airflow distribution within the holder. The holder may also include a cover 11, with negative and positive tips that feature holes to facilitate potential transfer throughout the batteries and supercapacitors within the holder.

The collector frame and battery holder can be constructed from a variety of materials, including stainless steel, aluminum, carbon steel, glass-lined steel, nickel alloys (e.g., hastelloy, monel), titanium, reinforced concrete, inconel (nickel-chromium alloy), polymers (e.g., polytetrafluoroethylene (PTFE), polyvinylidene fluoride (PVDF), polyethylene (PE), or polypropylene (PP)), and borosilicate glass. The modular generator can also be designed in various geometrical shapes, such as cylindrical, cubic, triangular, rectangular, square, pentagonal, hexagonal, heptagonal, octagonal, and trapezoidal, to accommodate different system requirements and design preferences.

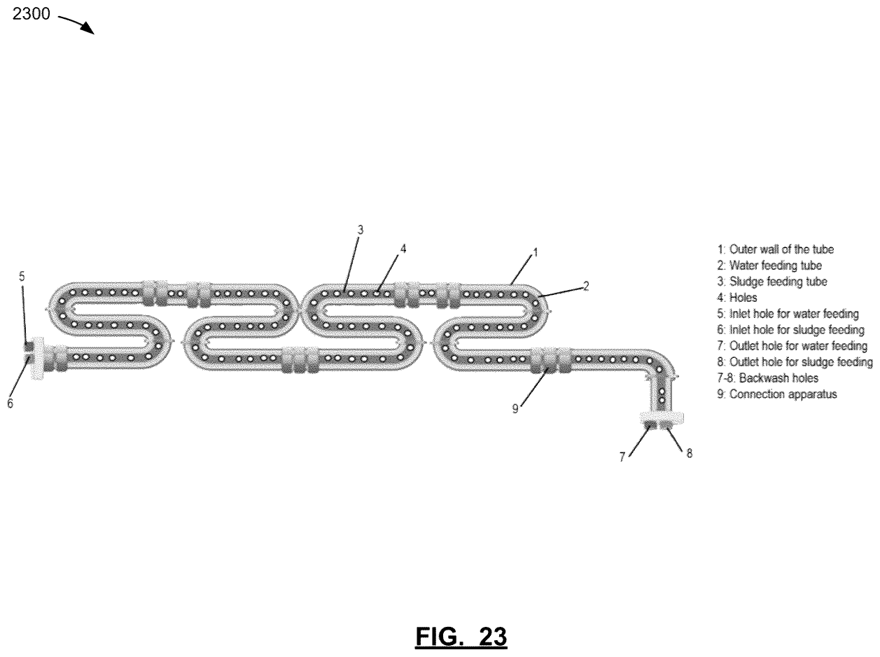

Reference is now made to FIG. 23, which illustrates an embodiment of a modular water and sludge feeding unit 2300 configured with cylindrical intertwined pipes. In this embodiment, the feeding unit 2300 is configured to provide the homogeneous mixing of two different solutions. The design provides microbial activity and promotes the interaction of microorganisms with water resources before the mixture is fed into the fermentation chamber or other types of liquid-comprising chambers.

The feeding unit 2300 can be configured in both horizontal and vertical orientations, providing flexibility in installation and operation. The sludge is introduced into the system through a perforated pipe located in the innermost part of the feeding unit 2300. As the sludge is fed into the system, it creates a biofilm within the pipe, increasing the effectiveness of the microorganisms. The holes 4 in the pipe provide that the water and sludge begin to interact within the feeding unit 2300 before being directed into the system.

Additionally, backwashing may be performed at regular intervals through the outlet holes of the modular feeding unit 2300. The process prevents the formation of excess biofilm that could cause clogging within the pipes, thereby maintaining the efficiency of the unit.

The frame of the feeding unit 2300 can be constructed using a variety of materials, including stainless steel, aluminum, carbon steel, glass-lined steel, nickel alloys such as hastelloy and monel, titanium, reinforced concrete, inconel (a nickel-chromium alloy), or polymers like polytetrafluoroethylene (PTFE), polyvinylidene fluoride (PVDF), polyethylene (PE), and polypropylene (PP). Borosilicate glass can also be used in the construction of the unit frame, providing durability and resistance to various environmental factors.

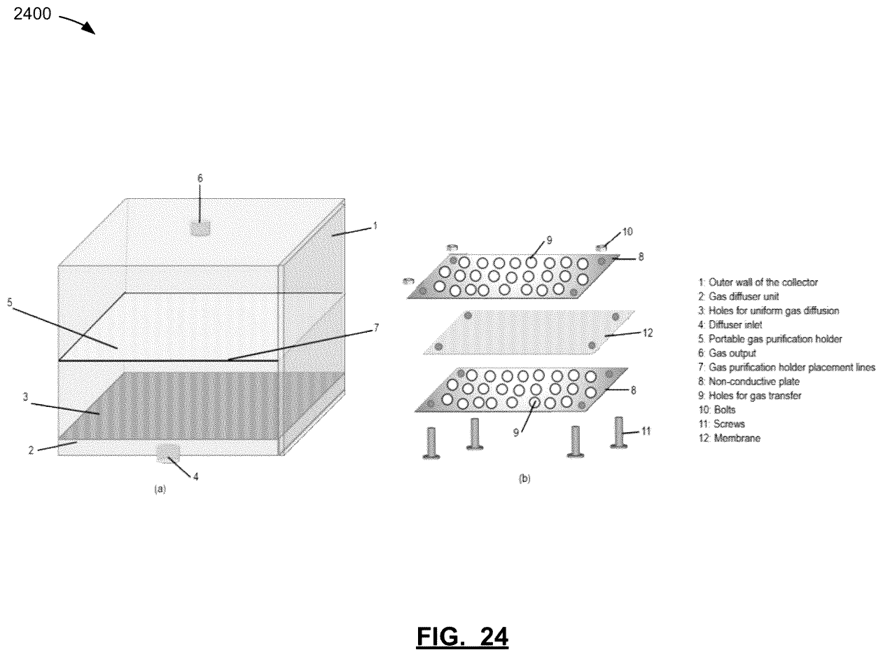

Reference is now made to FIG. 24A and FIG. 24B, which illustrate an embodiment of a modular hydrogen gas collector 2400 with an integrated modular gas purifier unit. In this embodiment, the hydrogen gas collector 2400 is designed to efficiently collect and purify hydrogen gas produced within a generator system.

The gas collector 2400 includes a modular gas purifier unit and an air diffuser. The air diffuser is positioned at the bottom of the collector chamber and is designed with holes to provide homogeneous gas distribution throughout the chamber. A non-conductive and corrosion-resistant apparatus is positioned above the air diffuser, that serves as a membrane holder. The apparatus is placed on specially designed lines within the chamber and is configured to prevent gas leakage from the sides, ensuring efficient gas collection and purification.

At the top of the collector 2400, a gas collection port 6 is provided for the further utilization of the purified hydrogen gas. The modular design of the gas purifier unit allows for flexibility in its application, and multiple gas purifier units can be placed within the collector 2500 if required by the specific application.

The gas purifier holder, as illustrated in FIG. 24B, comprises of non-conductive and corrosion-resistant plates 8 positioned at the top and bottom of the purifier unit. The plates 8 comprise of holes to facilitate interaction between the purifier reagent and the collected gas. The purifier material can be placed in the middle of these plates, forming a cascaded gas purifier unit. To provide a leak-proof operation, the main components of the purifier unit are clamped together with O-rings, bolts, and screws.

The modular design provides the gas collector to be used with or without the gas purifier unit, depending on the application requirements. The collector 2400 and its components can be constructed from various materials, including stainless steel, aluminum, carbon steel, glass-lined steel, nickel alloys such as hastelloy and monel, titanium, reinforced concrete, inconel (a nickel-chromium alloy), polymers such as polytetrafluoroethylene (PTFE), polyvinylidene fluoride (PVDF), polyethylene (PE), polypropylene (PP), and borosilicate glass.

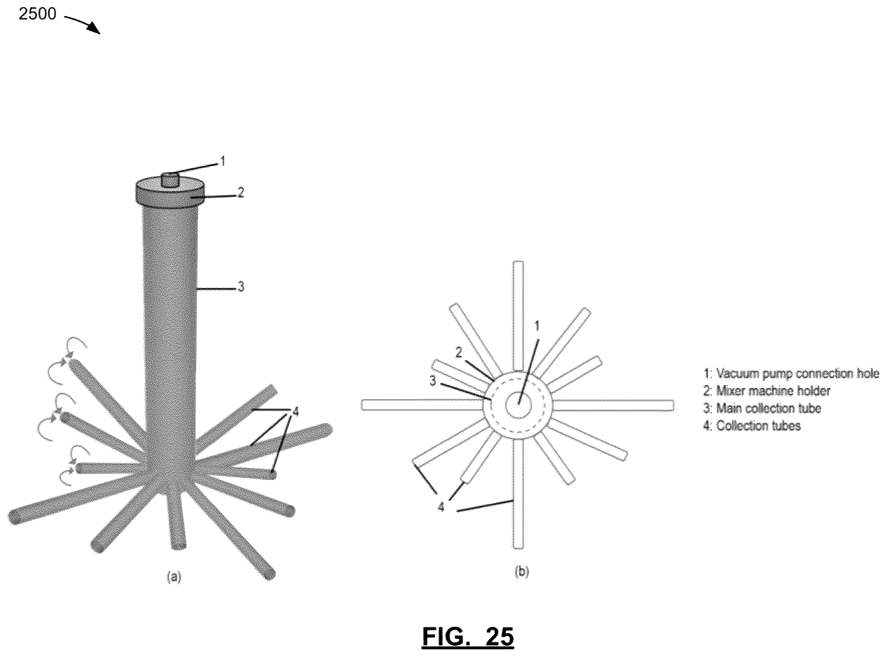

Reference is now made to FIG. 25A and FIG. 25B, which illustrates an embodiment of a modular sludge collection and mixing device 2500 designed for the effective collection and mixing of waste sludge within a fermentation chamber. In this embodiment, the sludge collection and mixing device 2500 may be used to further utilize waste sludge as fertilizer and to provide a homogeneous mixture within the chamber.

The sludge collection and mixing device 2500 comprises of a main pipe, which is connected to a plurality of external pipes. The external pipes are configured to collect excess sludge from the bottom of the fermentation chamber. The varying lengths of the external pipes provide for minimizing dead zones within the chamber, thereby ensuring effective sludge collection. The external pipes are connected to the main pipe 4, which operates as the central collection point.

Additionally, the device 2500 is configured to allow an external vacuum pump to be connected via a hole placed at the top of the main pipe. The connection enables the vacuum pump to assist in the collection of sludge. The main pipe may also include a mixer machine holder, for homogeneous mixing of the sludge within the chamber. Therefore, the modular device 2600 can function as both a mixer and a collector, to improve the overall efficiency of the fermentation process.

The collector and mixer device 2500 can be constructed using a variety of materials, including stainless steel, aluminum, carbon steel, glass-lined steel, nickel alloys such as hastelloy and monel, titanium, inconel (a nickel-chromium alloy), or polymers like polytetrafluoroethylene (PTFE), polyvinylidene fluoride (PVDF), polyethylene (PE), and polypropylene (PP). Borosilicate glass can also be used in the construction of the device, providing durability and resistance to various environmental factors.

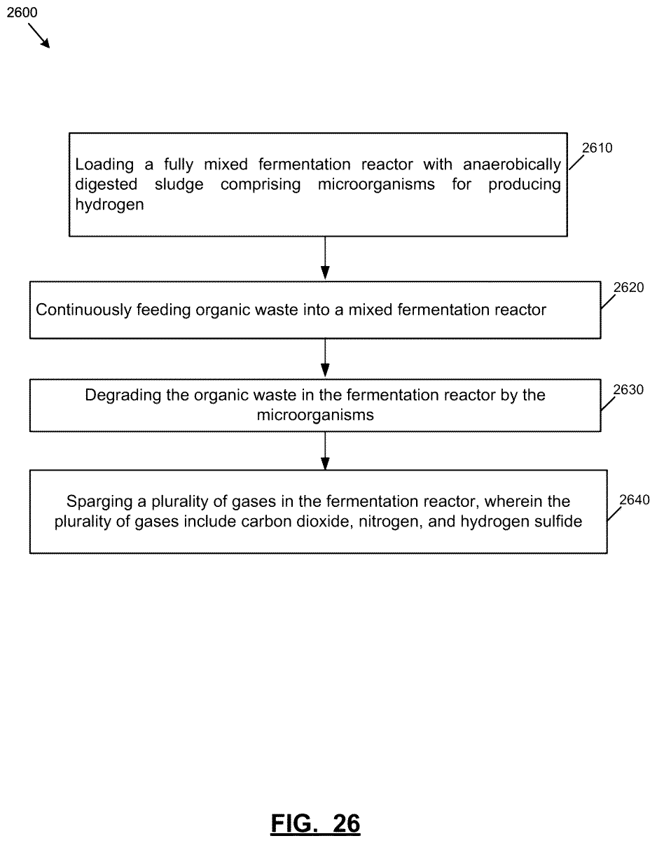

Reference is now made to FIG. 26, which shows a flowchart 2600 of an example method for continuously producing natural hydrogen gas in a fermentation chamber from organic waste and anaerobic microorganism resources. The method 2600 may be implemented by an integrated modular hydrogen production system, which utilizes anaerobic digestion, continuous feeding mechanisms, and gas sparging techniques to optimize hydrogen production.

At step 2610, the method includes loading a fully mixed fermentation reactor with anaerobically digested sludge. This sludge may be combined with a variety of microorganisms, some of which are capable of producing hydrogen. The process of loading the reactor may include blending the anaerobic sludge with selected microorganisms that are specifically chosen for their hydrogen-producing capabilities. The mixture is introduced into the fermentation reactor under controlled conditions to ensure uniform distribution of microorganisms throughout the sludge.

At step 2620, the method includes continuously feeding organic waste into the completely mixed fermentation reactor. This step provides that the fermentation process is sustained over time, providing a steady supply of organic material for the microorganisms to degrade. The organic waste may be introduced through an automated feeding system that maintains a consistent flow of waste into the reactor. The continuous feeding mechanism is designed to prevent interruptions in the fermentation process, thereby maximizing hydrogen production. Various types of organic waste, such as agricultural residues, food waste, and industrial by-products, may be used as feedstock, depending on availability and the specific requirements of the system.

At step 2630, the method includes using the hydrogen-producing bacteria in the anaerobic sludge to constantly degrade the organic waste in the fermentation reactor. The degradation process results in the production of hydrogen gas, as well as liquid effluents comprising of a mixture of alcohols, volatile fatty acids, and bacteria. The anaerobic sludge also comprises a mixture of organic substances and bacteria that contribute to the overall fermentation process. The continuous degradation of organic waste by the hydrogen-producing bacteria provides that hydrogen production is sustained over time. The method may include monitoring and adjusting the conditions within the reactor, such as pH, temperature, and retention time, to optimize the activity of the hydrogen-producing bacteria and improve the overall efficiency of the process.

At step 2640, the method includes continuously sparging various gases, such as carbon dioxide, nitrogen, and hydrogen sulfide, into the fermentation reactor. The sparging process enhances hydrogen production by providing a homogeneous mixture within the reactor and improving the microbial activity. The introduction of the gases may be controlled through a series of valves and diffusers that ensure even distribution throughout the reactor. Sparging maintains optimal conditions for the microorganisms, prevents the formation of gas pockets that could disrupt the fermentation process, and enhances the overall efficiency of hydrogen production. Additionally, the sparging process may be adjusted based on real-time data collected from sensors within the reactor, allowing for dynamic control of gas concentrations and flow rates.

In an embodiment, the method includes loading a fully mixed fermentation reactor with a biocatalyst that has been synthesized with a variety of microorganisms, algae, and fungi. The biocatalyst may be specifically engineered to enhance the efficiency of the fermentation process, leveraging the unique metabolic pathways of the included microorganisms, algae, and fungi. The combination of these biological agents may provide more effective degradation of organic material, leading to increased hydrogen production. The method may also include steps to ensure the even distribution of the biocatalyst within the reactor, optimizing contact between the biocatalyst and the organic waste.

In an embodiment, the method includes adjusting the ratio of organic waste to anaerobic sludge within the fermentation reactor. The ratio may be varied within a range from 1:1, 5:1, 10:1, 50:1 to 1:5, 1:10, and 1:20, depending on the specific requirements of the hydrogen production process. Adjusting the ratio may involve measuring and controlling the input of both organic waste and sludge to achieve the desired balance. The chosen ratio can improve the efficiency of the fermentation process, with ratios favoring higher hydrogen yields or faster processing times.

In an embodiment, the method includes controlling the temperature of the fermentation reactor. The temperature within the reactor may be maintained within a range from approximately 20° C. to nearly 55° C. Controlling the temperature provides for optimizing microbial activity, as different microorganisms have specific temperature ranges where they function most effectively. The method may include continuously monitoring the reactor's temperature and making adjustments as needed to ensure it remains within the optimal range for hydrogen production.

In an embodiment, the method includes adjusting the temperature of the fermentation reactor to the desired values using various heating mechanisms. The mechanisms may include a heating plate, induction heating coil, heating tape, jacketed reactors, heating blankets, immersion heaters, heat exchangers, and infrared heaters. The method may also include selecting the appropriate heating method based on factors such as the size of the reactor, the thermal properties of the materials involved, and the specific temperature requirements of the fermentation process. The goal is to maintain a stable and uniform temperature throughout the reactor to promote efficient microbial activity.

In an embodiment, the method includes controlling the pH of the fermentation reactor. The pH may be maintained within a range from approximately 3 to nearly 9, depending on the optimal conditions for the specific microorganisms involved in the hydrogen production process. Controlling the pH provides for maintaining the stability of the microbial environment and preventing conditions that could inhibit hydrogen production. The method may involve regular monitoring of the pH levels and making necessary adjustments to keep the pH within the desired range.

In an embodiment, the method includes adjusting the pH of the fermentation reactor to desired values using various chemical agents. These agents may include sodium hydroxide, sodium bicarbonate, calcium hydroxide, magnesium hydroxide, hydrochloric acid, sulfuric acid, nitric acid, and acetic acid. The selection of the appropriate chemical agent may depend on the existing pH level and the specific requirements of the fermentation process. The method may also include controlling the dosage of these chemicals to achieve precise pH adjustments without causing adverse effects on the microorganisms or the overall process.

In an embodiment, the method includes placing anode and cathode electrodes within the fermentation reactor to collect bioenergy produced by microorganisms during the degradation of organic substances. The electrodes may be composed of various materials, including carbon, copper, chromium, cadmium, bronze, germanium, graphite, gold, nickel, zinc, lead, platinum, titanium, rhodium, palladium, iridium, ruthenium, and silver. These electrodes may be shaped in various forms such as plates, scraps, particles, or brushes to maximize the surface area available for microbial activity and bioenergy collection. The method may also involve optimizing the placement and configuration of the electrodes to enhance the efficiency of bioenergy collection.

In an embodiment, the method includes continuously collecting hydrogen gas, which is recovered from the headspace of the fermentation reactor. The method may involve using specialized gas collection systems that ensure the efficient and uninterrupted capture of hydrogen as it is produced. This continuous collection process provides for maintaining the overall efficiency of the hydrogen production system and preventing the buildup of gases that could inhibit the fermentation process. The collected hydrogen may then be stored or utilized in subsequent processes, depending on the specific application of the system.

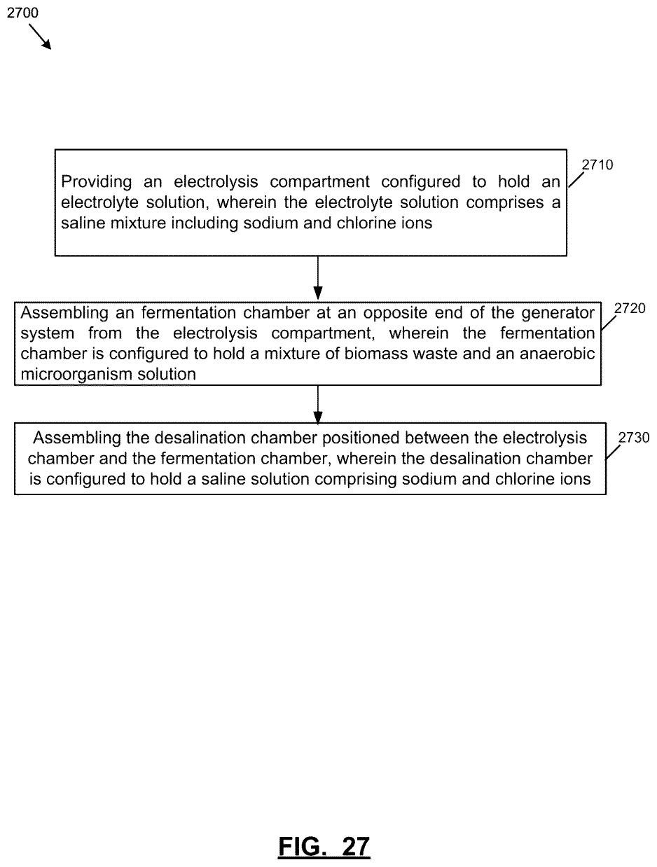

Reference is now made to FIG. 27, which illustrates an example method 2700 for assembling a modular three-chambered cylindrical natural hydrogen, freshwater, and bioelectricity generator system 100 as shown in FIG. 1.

At step 2710, the method includes providing the electrolysis compartment configured to hold an electrolyte solution. The electrolyte solution, which may comprise a saline mixture including sodium, chlorine ions, and other anions and cations, is introduced into the chamber. The method may further include positioning first and second electrodes within the electrolyte solution, providing they are spaced apart and at least partially submerged. Holes may be bored into these electrodes, and conductive wires are wound around them. The electrodes are then fixed in place using the holes located at the top of the electrolysis compartment. Additionally, the method may include connecting the electrodes to an external electrical circuit through an applied external resistance. To facilitate the flow of the electrolyte solution, inlet and outlet holes are formed at the top of the electrolysis chamber, and a hole 1 is made for hydrogen gas collection.

At step 2720, the method includes assembling the fermentation chamber at the opposite end of the generator system from the electrolysis compartment. The fermentation chamber is configured to hold a mixture of biomass waste and an anaerobic microorganism solution comprising organic substances and various types of bacteria. The method may include inserting first and second spaced apart electrodes into the fermentation chamber, ensuring they are partially immersed in the solution to facilitate bioenergy collection. Holes may be bored into these electrodes, and conductive wires are wound around them. The electrodes may be then secured in place using the holes at the top of the fermentation chamber. The method also includes connecting the electrodes to an external multimeter to measure the potential produced within the chamber. Additionally, a hole is made at the top of the fermentation chamber for hydrogen gas collection.

At step 2730, the method includes assembling the desalination chamber positioned between the electrolysis chamber and the fermentation chamber. The desalination chamber is configured to hold a saline solution comprising sodium, chlorine ions, and other anions and cations. The method may include installing an anion exchange membrane and a cation exchange membrane to separate the desalination chamber from the electrolysis and fermentation chambers, respectively. The membranes provide for ion exchange processes necessary for desalination. To provide leak-proof operation, the method includes clamping the electrolysis chamber, desalination chamber, and fermentation chamber together using gaskets and O-rings, secured by stainless steel bolts.

According to an embodiment, the method may further include installing mixers within the generator system. These mixers, which may be of paddle-type, spiral, or ribbon design, are placed horizontally within both the fermentation chamber and the electrolysis chamber. The mixers are essential for maintaining a homogeneous solution within each chamber, contributing to the overall efficiency and performance of the generator system.

According to an embodiment, the method may further include the integration of all three compartments electrolysis, desalination, and fermentation into a unified modular structure. This step may include securing the modular structure to ensure stability and alignment of the components. The method may also include connecting the external circuits and gas collection systems, providing that all necessary seals are in place.

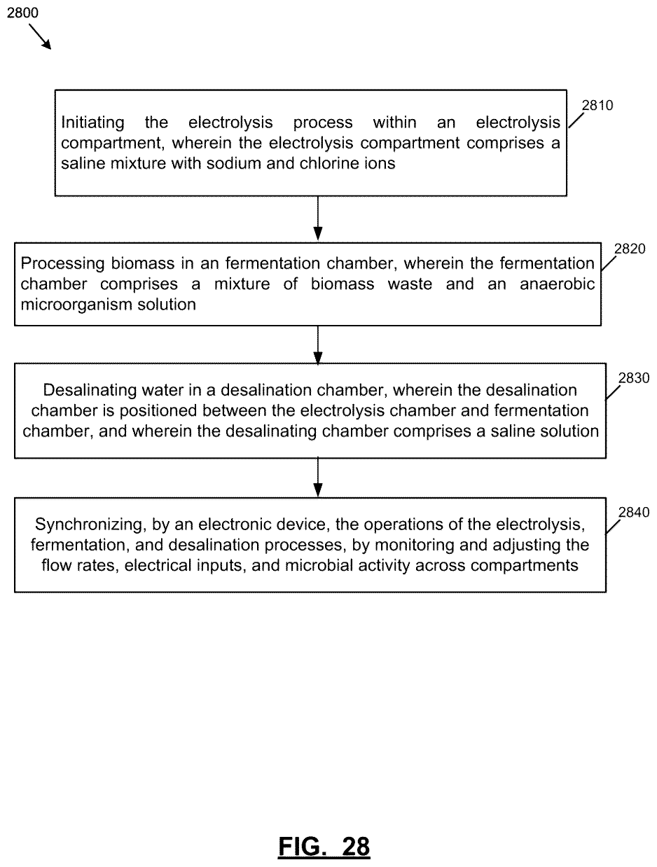

Reference is now made to FIG. 28, which illustrates an example method 2800 for generating electricity, desalinating water, and fermenting biomass within a modular three-chambered cylindrical natural hydrogen, freshwater, and bioelectricity generator system.

At step 2810, the method includes initiating the electrolysis process within the electrolysis compartment, wherein the electrolysis compartment comprises a saline mixture with sodium, chlorine ions, and other anions and cations. First and second electrodes which are spaced apart and submerged in the electrolyte solution, are connected to an external electrical circuit through an applied external resistance. The passage of an electric current through the electrolyte solution initiates the electrochemical reaction, generating hydrogen gas at the cathode and oxygen gas at the anode. The hydrogen gas produced in this process is collected for use, while the electrical energy generated contributes to the bioelectricity output of the system.

At step 2820, the method includes processing biomass in the fermentation chamber. The fermentation chamber is loaded with a mixture of biomass waste and an anaerobic microorganism solution. The bacteria within this solution, including hydrolytic, acidogenic, acetogenic, and methanogenic bacteria, work synergistically to break down complex organic materials in the biomass. This microbial activity produces hydrogen gas, along with a mixture of alcohols, volatile fatty acids, and bacteria. The hydrogen gas is collected from the fermentation chamber, contributing to the overall hydrogen production of the system, while the bioelectricity generated through the microbial degradation process is captured by electrodes placed within the fermentation chamber.

At step 2830, the method includes desalinating water in the desalination chamber. The desalination chamber, positioned between the electrolysis and fermentation chambers, comprises a saline solution similar to that in the electrolysis chamber. Anion exchange membrane and cation exchange membrane separate the desalination chamber from the electrolysis and fermentation chambers, respectively. The method includes applying an electrical field across the membranes, which drives the ion exchange process, removing sodium and chlorine ions from the saline solution and producing freshwater. The desalinated water is collected for use, contributing to the system's freshwater output.

At step 2840, the method includes synchronizing the operations of the electrolysis, fermentation, and desalination processes. This synchronization may include providing an electronic device for monitoring and adjusting the flow rates, electrical inputs, and microbial activity across the compartments. The method may also include real-time feedback control mechanisms to maintain the desired balance between hydrogen production, bioelectricity generation, and freshwater output.

Numerous specific details are set forth herein in order to provide a thorough understanding of the exemplary embodiments described herein. However, it will be understood by those of ordinary skill in the art that these embodiments may be practiced without these specific details. In other instances, well-known methods, procedures and components have not been described in detail so as not to obscure the description of the embodiments. Furthermore, this description is not to be considered as limiting the scope of these embodiments in any way, but rather as merely describing the implementation of these various embodiments.

1. A modular three-chambered cylindrical generator system, comprising:
a fermentation compartment configured to receive a mixture of biomass waste and an anaerobic microorganism solution comprising bacteria for bioenergy production, wherein the fermentation compartment includes first and second spaced apart electrodes at least partially immersed in the mixture;
an electrolysis compartment configured to receive an electrolyte solution comprising a saline mixture, the electrolysis compartment including first and second spaced apart electrodes at least partially submerged in the electrolyte solution, the first and second spaced apart electrodes connected to an external electrical circuit through an applied external resistance; and
a desalination compartment positioned between the fermentation compartment and the electrolysis compartment, the desalination compartment configured to receive a saline solution and comprising an anion exchange membrane separating the desalination compartment from the electrolysis compartment and a cation exchange membrane separating the desalination compartment from the fermentation compartment, wherein the desalination compartment is configured to perform ion exchange processes to produce freshwater.

2. The generator system of claim 1, wherein the first and second electrodes in the electrolysis compartment are spaced apart by a distance of approximately 5 to 10 centimeters.

3. The generator system of claim 1, wherein the electrodes in the fermentation compartment are connected to an external multimeter configured to measure the bioelectricity potential.

4. The generator system of claim 1, further comprising mixers selected from the group consisting of paddle-type mixers, spiral mixers, and ribbon mixers, wherein the mixers are placed horizontally within the fermentation compartment and the electrolysis compartment.

5. The generator system of claim 1, wherein the fermentation compartment, the desalination compartment, and the electrolysis compartment are clamped together using gaskets and O-rings secured by stainless steel bolts to provide a leak-proof operation.

6. A modular cascaded generator system, comprising:
an electrolysis compartment configured to receive an electrolyte solution comprising a saline mixture, the electrolysis compartment including first and second electrodes spaced apart and positioned parallel to each other on movable rails, the first and second electrodes connected to an external electrical circuit through an applied external resistance;
a fermentation compartment configured to receive a mixture of biomass waste and an anaerobic microorganism solution comprising bacteria for bioenergy production, wherein the fermentation compartment includes first and second electrodes spaced apart and positioned parallel to each other on movable rails, the first and second electrodes connected to an external multimeter for measuring bioelectricity potential; and
a desalination compartment positioned between the electrolysis compartment and the fermentation compartment, the desalination compartment configured to receive a saline solution and comprising an anion exchange membrane separating the desalination compartment from the electrolysis compartment and a cation exchange membrane separating the desalination compartment from the fermentation compartment, wherein the desalination compartment is configured to perform ion exchange processes to produce freshwater;
wherein the compartments are clamped together using gaskets and O-rings secured by stainless steel bolts to provide a leak-proof operation.

7. The generator system of claim 6, wherein the first and second electrodes in the electrolysis compartment are spaced apart by a distance of approximately 5 to 10 centimeters.

8. The generator system of claim 6, wherein the electrolysis compartment further comprises multiple electrodes placed within the electrolysis compartment.

9. The generator system of claim 6, wherein the fermentation compartment is located above the desalination compartment and the hydrogen gas produced in the fermentation compartment is collected through a hole positioned at the top of the fermentation compartment.

10. The generator system of claim 6, wherein the desalination compartment includes an input located at the top of the desalination compartment for solution feeding and an output located at the bottom of the desalination compartment for solution collection.

11. A modular intertwined generator system for the concurrent production of natural hydrogen, freshwater, and bioelectricity, comprising:
an electrolysis chamber located in an interior of the generator system, the electrolysis chamber including cylindrical electrodes spaced apart and separated from a desalination chamber by a cation exchange membrane, the cylindrical electrodes connected to an external electrical circuit via an applied external resistance, wherein the electrolysis chamber is configured to receive an electrolyte solution and hydrogen gas produced during the electrolysis process;
a desalination chamber positioned in the middle of the generator system, the desalination chamber separated from the electrolysis chamber by the cation exchange membrane and from a fermentation chamber by an anion exchange membrane, the desalination chamber configured to receive a saline solution; and
a fermentation chamber located at the outermost part of the generator system, the fermentation chamber including first and second spaced apart electrodes placed in a biomass solution for bioenergy collection, the electrodes connected to an external multimeter for measuring the bioelectricity potential.

12. The generator system of claim 11, wherein the cylindrical electrodes in the electrolysis chamber are spaced apart by a distance of approximately 5 to 10 centimeters.

13. The generator system of claim 12, wherein the generator system is constructed in a geometrical shape selected from a group of cylindrical, cubic, triangular, rectangular, square, pentagonal, hexagonal, heptagonal, octagonal, and trapezoidal.

14. A method for generating electricity, desalinating water, and fermenting biomass within a modular three-chambered generator system, the method comprising:
initiating an electrolysis process within an electrolysis compartment to generate hydrogen gas and bioelectricity, wherein the electrolysis compartment comprises a saline mixture with sodium and chlorine ions;
processing biomass in a fermentation chamber to produce hydrogen gas and bioelectricity, wherein the fermentation chamber comprises a mixture of biomass waste and an anaerobic microorganism solution;
desalinating water in a desalination chamber positioned between the electrolysis chamber and fermentation chamber, wherein the desalinating chamber comprises a saline solution; and
synchronizing, by an electronic device, the electrolysis, fermentation, and desalination processes, by monitoring and adjusting the flow rates, electrical inputs, and microbial activity across compartments.
